(19) United States
(12) Patent Application Publication
(10) Pub. No.: US 2022/0118217 A1
(43) Pub. Date: Apr. 21, 2022

(54) DEVICES AND METHODS FOR USING MECHANICAL AFFECTIVE TOUCH THERAPY TO IMPROVE FOCUS, CONCENTRATION, LEARNING CAPACITY, VISUAL MEMORY, NEW LEARNING, SUSTAINED ATTENTION, COGNITION & INTEROCEPTION IN HUMANS

(71) Applicant: Apex Neuro Holdings, Inc., Brooklyn, NY (US)

(72) Inventors: Francois Kress, New York, NY (US); Sean Hagberg, Cranston, RI (US); Durga Sahithi Garikapati, Bangalore (IN); Gina Sensale, Andover Township, NJ (US)

(21) Appl. No.: 17/566,570

(22) Filed: Dec. 30, 2021

Related U.S. Application Data
(63) Continuation-in-part of application No. 17/026,268, filed on Sep. 20, 2020.

(51) Int. Cl. A61M 21/00; G09B 19/00

(52) U.S. Cl. CPC A61M 21/00; G09B 19/00; A61M 2209/088; A61M 2205/8206; A61M 2021/0022

(57) ABSTRACT

Methods and devices that improve focus, concentration, learning capacity, visual memory, new learning, sustained attention, cognition and/or interoception in a human using mechanical affective touch therapy are provided. In one embodiment, the method comprises delivering to a human body transcutaneous mechanical vibrations having a frequency of less than 20 Hz for at least 10 minutes, at least 2 times per day, for a period of at least 4 weeks, thereby providing the human with transcutaneous mechanical stimulation that improves focus, concentration, learning capacity, visual memory, new learning, sustained attention, cognition and interoception in that human.

### PRIORITY

This application hereby claims priority to and benefit of U.S. Non-Provisional patent application Ser. No. 17/026,268, filed on Sep. 20, 2020, the contents of which is hereby incorporated by reference herein in its entirety.

### FIELD OF THE DISCLOSURE

The present disclosure relates generally to wearable devices and associated methods that provide a variety of health benefits to mammals. In particular, the various embodiments of the devices and associated methods disclosed herein show a significant improvement in focus, concentration, learning capacity, visual memory, new learning, sustained attention, cognition and interoception in humans using the mechanically affective touch therapy devices and associated methods disclosed herein.

### GENERAL BACKGROUND

Interoception is the process by which your brain interprets what is going on in any given area of your body, some of which you may not always be aware of. This information can then be integrated into other processes, influencing how we think, perceive, and process information. Elite athletes, successful CEOs, bond traders, hostage negotiators and snipers, among others are known to have greater interoceptive perception and accuracy. Accordingly, it is desirable to increase interoception in a human. The present disclosure provides methods and devices using affective touch to provide a variety of benefits to a human. In particular, as demonstrated by the data disclosed herein, the methods and devices disclosed herein provide a significant improvement in focus, concentration, learning capacity, visual memory, new learning, sustained attention, cognition and/or interoception in humans.

### SUMMARY

Presented herein are methods and devices that improve focus, concentration, learning capacity, visual memory, new learning, sustained attention, cognition and interoception using mechanical affective touch therapy (MATT). In certain embodiments, the approaches described herein utilize a stimulation device (e.g., a wearable or applied device) for generation and delivery of the affective touch therapy, which in at least some of the embodiments disclosed herein, is provided through mechanical vibrational waves.

As described herein, the delivered vibrational waves can be tailored based on particular targets (e.g., nerves, mechanoreceptors, vascular targets, tissue regions) to stimulate and/or to elicit particular desired responses in a subject. As described herein, in certain embodiments, the delivery of mechanical stimulation to a human improve focus, concentration, learning capacity, visual memory, new learning, sustained attention, cognition and interoception in that human.

In certain embodiments, the properties of mechanical waves generated are tailored by controlling a waveform of an electronic drive signal that is applied to mechanical transducers in order to generate a desired mechanical wave. By controlling and delivering various specific mechanical waves in this manner, the approaches described herein can be used to achieve a variety of health benefits in subjects, for example and not by way of limitation, improving focus, concentration, learning capacity, visual memory, new learning, sustained attention, cognition and/or interoception.

In at least one embodiment of this disclosure, a device for improving focus, concentration, learning capacity, visual memory, new learning, sustained attention, cognition and interoception in a human is provided. In one embodiment, the device comprises (1) one or more mechanical transducers, (2) one or more batteries, (3) one or more sinusoidal waveforms and (4) one or more controller boards that control at least the one or more sinusoidal waveforms output through the mechanical transducers. The one or more mechanical transducers, batteries and controller boards are in communication with each other. The controller board controls the sinusoidal waveform output through the one or more mechanical transducers, thereby producing mechanical vibrations for a human. The device is adapted to provide mechanical vibrations in proximity to the temporal bone of the human's head.

In at least one aspect of at least one embodiment of this disclosure, the frequency of the one or more waveform is less than 20 Hz.

In another aspect of at least one embodiment of this disclosure, the frequency of the one or more waveforms is approximately 10 Hz.

In another aspect of at least one embodiment of this disclosure, the one or more waveforms are isochronic.

In another aspect of at least one embodiment of this disclosure, the isochronic waveforms are provided for a period of about two seconds.

In another aspect of at least one embodiment of this disclosure, the device delivers mechanical vibrations in proximity to the temporal bone for at least 10 minutes per day.

In another aspect of at least one embodiment of this disclosure, the device delivers mechanical vibrations in proximity to the temporal bone at least one time per day for a period of at least 4 weeks.

In at least one other embodiment of the present disclosure, a method of improving focus, concentration, learning capacity, visual memory, new learning, sustained attention, cognition and/or interoception in a human is provided. The method comprises (1) generating mechanical vibrations using a mechanical transducer of a transcutaneous mechanical stimulation device in response to an applied electronic drive signal, (2) controlling the mechanical vibrations of the electronic drive signal by a controller board so that the mechanical vibrations have a frequency of less than 20 Hz; and (3) delivering the mechanical vibrations to the body of the human via the mechanical stimulation device, thereby providing the human with transcutaneous mechanical stimulation that improves focus, concentration, learning capacity, visual memory, new learning, sustained attention, cognition and/or interoception.

In at least another aspect of at least one embodiment of the disclosure, the mechanical vibrations are provided to the C-tactile afferents in the human hairy skin.

In at least another aspect of at least one embodiment of the disclosure, the device delivers mechanical vibrations to the humans head area at least 20 minutes per day.

In at least another aspect of at least one embodiment of the disclosure, the device delivers mechanical vibrations to the humans head area at least 2 times per day.

In another embodiment of the present disclosure, a device for improving focus, concentration, learning capacity, visual memory, new learning, sustained attention, cognition and interoception is provided. The device comprises one or more mechanical transducers, one or more batteries and one or more controller boards, where the one or more mechanical transducers, the one or more batteries and the one or more controller boards are in communication and when the device's mechanical transducers provide mechanical vibrations on the human head.

In yet another embodiment of the present disclosure, a device for improving focus, concentration and learning capacity is provided. The device comprises one or more mechanical transducers, one or more batteries, and one or more controller boards, where the one or more mechanical transducers, the one or more batteries and the one or more controller boards are in communication and when the device's mechanical transducers provide mechanical vibrations near the human's head, the human's focus, concentration and learning capacity is improved.

In yet another embodiment of the present disclosure, a method of improving visual memory, new learning and sustained attention in a human is provided. The method comprises (1) generating mechanical vibrations using a mechanical transducer of a transcutaneous mechanical stimulation device, (2) controlling the mechanical vibrations of the electronic drive signal by a controller board so that the mechanical vibrations have a frequency of less than 20 Hz and (3) delivering the mechanical vibrations to the body of the human via the mechanical stimulation device, thereby providing the human with transcutaneous mechanical stimulation that improves visual memory, new learning and sustained attention.

In yet another aspect of at least one embodiment, the disclosure is directed to a transcutaneous neuromodulation device [e.g., a wearable device; e.g., a non-invasive device (e.g., not comprising any components that penetrate skin)] for improving focus, concentration, learning capacity, visual memory, new learning, sustained attention, cognition and/or interoception in a human by promoting nerve stimulation through mechanical vibration, comprising: one or more mechanical transducers, a battery, and one or more controller boards, wherein the one or more mechanical transducers, the battery and the one or more controller boards are in communication (e.g., through one or more connectors or wirelessly), and wherein the controller board controls waveform output through the one or more mechanical transducers, thereby producing mechanical vibration.

In certain embodiments, the one or more nerves comprising a C-tactile afferent mechanoreceptor.

In certain embodiments, the device promotes stimulation of (e.g., wherein the waveform is selected to promote stimulation of) one or more mechanoreceptors and/or cutaneous sensory receptors in the skin (e.g., to stimulate an afferent sensory pathway and use properties of receptive fields to propagate stimulation through tissue and bone). In certain embodiments, the one or more mechanoreceptors and/or cutaneous sensory receptors comprise Piezo2 protein and/or Merkel cells.

In certain embodiments, the one or more controller boards modulate the waveform output to introduce particular signals that include active or inactive pulse durations and frequencies configured to accommodate particular mechanoreceptor recovery periods, adaptation times, inactivation times, sensitization and desensitization times, or latencies.

In certain embodiments, the one or more controller boards modulate the waveform output to enhance or inhibit the expression of presynaptic molecules essential for synaptic vesicle release in neurons.

In certain embodiments, the one or more controller boards modulate the waveform output to enhance or inhibit the expression of neuroactive substances that can act as fast excitatory neurotransmitters or neuromodulators.

In certain embodiments, the one or more controller boards modulates the waveform output to stimulate mechanoreceptor cell associated with Aδ-fibers and C-fibers (e.g., including C tactile fibers) in order to stimulate nociceptive, thermoceptive and other pathways modulated by these fibers.

In certain embodiments, the one or more controller boards modulate the waveform output using dynamical systems methods to produce a preferred response in neural network dynamics (e.g., via modulation of signal timing).

In certain embodiments, the one or more controller boards modulates the waveform output using dynamical systems measures to assess response signals (e.g., electronic) to detect particular network responses correlated with changes in mechanical wave properties (e.g., and modulates the waveform output to target/optimally enhance particular preferred responses).

In certain embodiments, the device comprises at least one transducer array comprising a plurality of (e.g., two or more) mechanical transducers maintained in a fixed spatial arrangement in relation to each other (e.g., in substantial proximity to each other; e.g., spaced along a straight or curved line segment) and wherein at least a portion of the one or more controller boards (e.g., a single controller board; e.g., two or more controller boards) are in communication with the mechanical transducers of the transducer array to control output of the mechanical transducers of the transducer array in relation to each other [e.g., wherein the at least a portion of the one or more controller boards synchronizes mechanical vibration produced by each mechanical transducer of the transducer array (e.g., such that each mechanical transducer begins and/or ends producing mechanical vibration at a particular delay with respect to one or more other mechanical transducers of the array; e.g., such that the mechanical transducers are sequentially triggered, one after the other; e.g., wherein the mechanical transducers are spaced along a straight or curved line segment and triggered sequentially along the line segment, such that an apparent source of mechanical vibration moves along the line segment to mimic a stroking motion)] [e.g., wherein a first portion of the mechanical transducers outputs a different frequency mechanical vibration from a second portion of the mechanical transducers of the transducer array (e.g., wherein each mechanical transducer of the transducer array outputs a different frequency mechanical vibration)].

In certain embodiments, the transducer is a linear transducer (e.g., operable to produce mechanical vibration comprising a longitudinal component (e.g., a longitudinal vibration)).

In certain embodiments, the device comprises a receiver in communication with the one or more controller boards, wherein the receiver is operable to receive a signal from and/or transmit a signal (e.g., wirelessly; e.g., via a wired connection) to a personal computing device (e.g., a smart phone; e.g., a personal computer; e.g., a laptop computer; e.g., a tablet computer; e.g., a smart watch; e.g., a fitness tracker; e.g., a smart charger, e.g., an app or remote storage)(e.g., to upload new waveforms and/or settings for waveforms).

In certain embodiments, the one or more controller boards is/are operable to modulate and/or select the waveform output in response to (e.g., based on) the signal received from the personal computing device by the receiver.

In certain embodiments, one or more low-amplitude sub-intervals of the isochronic wave have a duration of greater than or approximately two seconds (e.g., wherein the one or more low-amplitude sub-intervals have a duration of approximately two seconds; e.g., wherein the one or more low-amplitude sub-intervals have a duration ranging from approximately two seconds to approximately 10 seconds; e.g., wherein the one or more low amplitude sub-intervals have a duration ranging from approximately two seconds to approximately 4 seconds).

In certain embodiments, the isochronic wave comprises a carrier wave [e.g., a periodic wave having a substantially constant frequency (e.g., ranging from 0 to 20 Hz; or approximately 7 to approximately 13 Hz; e.g., a frequency range matching an alpha brain wave frequency range; e.g., approximately 10 Hz)] modulated by an envelope function having one or more low-amplitude sub-intervals [e.g., a periodic envelope function (e.g., a square wave; e.g., a 0.5 Hz square wave); e.g., the one or more low-amplitude sub-intervals having a duration of greater than or approximately equal to two seconds; e.g., the one or more low-amplitude sub-intervals having a duration of approximately two seconds].

In certain embodiments, the isochronic wave is also a transformed time-varying wave. In certain embodiments, the isochronic wave comprises a clipped or carrier wave. In certain embodiments, the waveform output comprises a transformed time-varying wave having a functional form corresponding to a carrier wave within an envelope {e.g., wherein the transformed-time varying wave is the carrier wave and is further modulated by an envelope [e.g., wherein the envelope is a sinusoidal wave; e.g., wherein the envelope has a monotonically increasing (in time) amplitude (e.g., wherein the envelope has a functional form corresponding to an increasing (in time) exponential)]; e.g., wherein the transformed time-varying wave is the envelope that modulates a carrier wave [e.g., wherein the carrier wave is a periodic wave (e.g., a sinusoidal wave; e.g., a square wave; e.g., a sawtooth wave)(e.g., having a higher frequency than the envelope)]}.

In certain embodiments, the device comprises a receiver in communication with the one or more controller boards, wherein the receiver is operable to receive a signal from and/or transmit a signal to a monitoring device (e.g., directly from and/or to the monitoring device; e.g., via one or more intermediate server(s) and/or computing device(s))(e.g., a wearable monitoring device; e.g., a personal computing device; e.g., a fitness tracker; e.g., a heart-rate monitor; e.g., an electrocardiograph (EKG) monitor; e.g., an electroencephalography (EEG) monitor; e.g., an accelerometer; e.g., a blood-pressure monitor; e.g., a galvanic skin response (GSR) monitor) and wherein the one or more controller boards is/are operable to modulate and/or select the waveform output in response to (e.g., based on) the signal from the wearable monitoring device received by the receiver.

In certain embodiments, the device is operable to record usage data (e.g., parameters such as a record of when the device was used, duration of use, etc.) and/or one or more biofeedback signals for a human subject [e.g., wherein the device comprises one or more sensors, each operable to measure and record one or more biofeedback signals (e.g., a galvanic skin response (GSR) sensor; e.g., a heart-rate monitor; e.g., an accelerometer)] [e.g., wherein the device is operable to store the recorded usage data and/or biofeedback signals for further processing and/or transmission to an external computing device, e.g., for computation (e.g., using a machine learning algorithm that receives the one or more biofeedback signals as input, along with, optionally, user reported information) and display of one or more performance metrics (e.g., a stress index) to a subject using the device].

In other embodiments, the one or more controller boards is/are operable to automatically modulate and/or select the waveform output in response to (e.g., based on) the recorded usage data and/or biofeedback signals (e.g., using a machine learning algorithm that receives the one or more biofeedback signals as input, along with, optionally, user reported information, to optimize the waveform output).

In other embodiments, a level [e.g., amplitude (e.g., a force; e.g., a displacement)] of at least a portion of the mechanical vibration is based on activation thresholds of one or more target cells and/or proteins (e.g., mechanoreceptors (e.g., C tactile afferents); e.g., nerves; e.g., sensory thresholds corresponding to a level of tactile sensation) [e.g., wherein the one or more controller boards modulate the waveform output based on sub-activation thresholds (e.g., accounting for the response of the mechanical transducers)].

In certain embodiments, an amplitude of the mechanical vibration corresponds to a displacement ranging from 1 micron to 10 millimeters (e.g., approximately 25 microns and in at least one embodiment 0.01 mm) (e.g., wherein the amplitude is adjustable over the displacement ranging from 1 micron to 10 millimeters) [e.g., wherein the amplitude corresponds to a force of approximately 0.4N] [e.g., thereby matching the amplitude to activation thresholds of C tactile afferents].

In certain embodiments, the isochronic wave comprises one or more components (e.g., additive noise; e.g., stochastic resonance signals) that, when transduced by the transducer to produce the mechanical wave, correspond to sub-threshold signals that are below an activation threshold of one or more target cells and/or proteins (e.g., below a level of tactile sensation).

In certain embodiments, the isochronic wave comprises one or more components (e.g., additive noise; e.g., stochastic resonance signals) that, when transduced by the transducer to produce the mechanical wave, correspond to supra-threshold signals that are above an activation threshold of one or more target cells and/or proteins (e.g., above a level of tactile sensation).

In another aspect, the disclosure is directed to a transcutaneous neuromodulation device [e.g., a wearable device; e.g., a non-invasive device (e.g., not comprising any components that penetrate skin)] for improving focus, concentration, learning capacity, visual memory, new learning, sustained attention, cognition and/or interoception in a human subject by promoting nerve stimulation through mechanical vibration, comprising: one or more mechanical transducers, a battery, and one or more controller boards, wherein the one or more mechanical transducers, the battery and the one or more controller boards are in communication (e.g., through one or more connectors; e.g., wirelessly), and wherein the one or more controller boards control waveform output through the one or more mechanical transducers, and the one or more mechanical transducers transcutaneously stimulate one or more nerves of a human subject and wherein the waveform output comprises an isochronic wave.

In another aspect, the disclosure is directed to a transcutaneous stimulation device [e.g., a wearable device; e.g., a non-invasive device (e.g., not comprising any components that penetrate skin)] for improving focus, concentration, learning capacity, visual memory, new learning, sustained attention, cognition and/or interoception in a human subject by promoting mechanoreceptor stimulation through mechanical vibration, comprising: one or more mechanical transducers, a battery, and one or more controller boards, wherein the one or more mechanical transducers, the battery and the one or more controller boards are in communication (e.g., through one or more connectors or wirelessly), and wherein the one or more controller boards control waveform output through the transducer, and the one or more mechanical transducers transcutaneously stimulate one or more mechanoreceptors of a human subject and wherein the waveform output comprises an isochronic wave.

In another aspect, the disclosure is directed to a method of improving focus, concentration, learning capacity, visual memory, new learning, sustained attention, cognition and/or interoception in a human by providing transcutaneous mechanical stimulation (e.g., non-invasive mechanical stimulation) to the subject via a stimulation device (e.g., a wearable device), the method comprising: generating a mechanical wave by a mechanical transducer of the stimulation device in response to an applied electronic drive signal; controlling a waveform of the electronic drive signal by a controller board (e.g., a controller board of the stimulation device; e.g., a remote controller board), wherein the waveform comprises an isochronic wave; and delivering the mechanical wave to a body location of the subject via the stimulation device, thereby providing the transcutaneous mechanical stimulation to the subject.

In certain embodiments, the mechanical wave promotes stimulation (e.g., wherein the waveform is selected to promote stimulation) of one or more nerves [e.g., a vagus nerve; e.g., a trigeminal nerve; e.g., peripheral nerves; e.g., a greater auricular nerve; e.g., a lesser occipital nerve; e.g., one or more cranial nerves (e.g., cranial nerve VII; e.g., cranial nerve IX; e.g., cranial nerve XI; e.g., cranial nerve XII)]. In certain embodiments, the one or more nerves comprises a vagus nerve and/or a trigeminal nerve. In certain embodiments, the one or more nerves comprises a C-tactile afferent.

In certain embodiments, the mechanical wave promotes stimulation of (e.g., wherein the waveform is selected to promote stimulation of) one or more mechanoreceptors and/or cutaneous sensory receptors in the skin (e.g., to stimulate an afferent sensory pathway and use properties of receptive fields to propagate stimulation through tissue and bone). In certain embodiments, the one or more mechanoreceptors and/or cutaneous sensory receptors comprise Piezo2 protein and/or Merkel cells.

In certain embodiments, the controlling of the waveform of the electronic drive signal comprises modulating the waveform to introduce particular signals that include active or inactive pulse durations and frequencies configured to accommodate particular mechanoreceptor recovery periods, adaptation times, inactivation times, sensitization and desensitization times, or latencies.

In certain embodiments, the controlling of the waveform of the electronic drive signal comprises modulating the waveform to enhance or inhibit the expression of presynaptic molecules essential for synaptic vesicle release in neurons.

In certain embodiments, the controlling of the waveform of the electronic drive signal comprises modulating the waveform to enhance or inhibit the expression of neuroactive substances that can act as fast excitatory neurotransmitters or neuromodulators.

In certain embodiments, the controlling of the waveform of the electronic drive signal comprises modulating the waveform to stimulate mechanoreceptor cells associated with Aδ-fibers and C-fibers (e.g., including C tactile fibers) in order to stimulate nociceptive, thermoceptive, interoceptive and/or other pathways modulated by these fibers.

In certain embodiments, controlling the waveform of the electronic drive signal comprises modulating the waveform using dynamical systems methods to produce a preferred response in neural network dynamics (e.g., via modulation of signal timing).

In certain embodiments, the controlling of the waveform of the electronic drive signal comprises modulating the waveform using dynamical systems measures to assess response signals (e.g., electronic) to detect particular network responses correlated with changes in mechanical wave properties (e.g., and modulates the waveform output to target/optimally enhance particular preferred responses).

In certain embodiments, delivering the mechanical wave to the body location comprises contacting the mechanical transducer to a surface (e.g., skin) of the subject at the body location.

In certain embodiments, the mechanical transducer is a member of a transducer array comprising a plurality of (e.g., two or more) mechanical transducers maintained in a fixed spatial arrangement in relation to each other (e.g., in substantial proximity to each other; e.g., spaced along a straight or curved line segment) and wherein the controller board controls output of the mechanical transducer in relation to other mechanical transducers of the array [e.g., so as to synchronize mechanical vibration produced by each mechanical transducer of the transducer array (e.g., such that each mechanical transducer begins and/or ends producing mechanical vibration at a particular delay with respect to one or more other mechanical transducers of the array; e.g., such that the mechanical transducers are sequentially triggered, one after the other; e.g., wherein the mechanical transducers are spaced along a straight or curved line segment and triggered sequentially along the line segment, such that an apparent source of mechanical vibration moves along the line segment to mimic a stroking motion)] [e.g., wherein a first portion of the mechanical transducers outputs a different frequency mechanical vibration from a second portion of the mechanical transducers of the transducer array (e.g., wherein each mechanical transducer of the transducer array outputs a different frequency mechanical vibration)].

In certain embodiments, the transducer is a linear transducer (e.g., operable to produce mechanical vibration comprising a longitudinal component (e.g., a longitudinal vibration)).

In certain embodiments, the mechanical transducer is incorporated into a headphone (e.g., an in-ear headphone; e.g., an over-the-ear headphone).

In certain embodiments, the controlling of the waveform of the electronic drive signal comprises receiving (e.g., by a receiver in communication with the controller board) a signal from a personal computing device (e.g., a smart phone; e.g., a personal computer; e.g., a laptop computer; e.g., a tablet computer; e.g., a smart watch; e.g., a fitness tracker; e.g., a smart charger)(e.g., to upload new waveforms and/or settings for waveforms).

In certain embodiments, the controlling of the waveform of the electronic drive signal comprises modulating and/or selecting the waveform in response to (e.g., based on) the signal received from the personal computing device by the receiver.

In certain embodiments, the delivering the mechanical wave to the body location is performed in a non-invasive fashion (e.g., without penetrating skin of the subject).

In certain embodiments, the method comprising providing, by a secondary stimulation device, one or more external stimulus/stimuli (e.g., visual stimulus; e.g., acoustic stimulus; e.g., limbic priming; e.g., a secondary tactile signal).

In certain embodiments, the isochronic wave comprises a frequency component ranging from 5 to 15 Hz (e.g., ranging from approximately 7 to approximately 13 Hz; e.g., a frequency range matching an alpha brain wave frequency range; e.g., approximately 10 Hz).

In certain embodiments, the isochronic wave comprises a frequency component ranging from 0 to 49 Hz (e.g., from 18 to 48 Hz; e.g., from 15 to 40 Hz; e.g. from 8 to 14 Hz).

In certain embodiments, one or more low-amplitude sub-intervals of the isochronic wave have a duration of greater than or approximately two seconds (e.g., wherein the one or more low-amplitude sub-intervals have a duration of approximately two seconds; e.g., wherein the one or more low-amplitude sub-intervals have a duration ranging from approximately two seconds to approximately 10 seconds; e.g., wherein the one or more low amplitude sub-intervals have a duration ranging from approximately two seconds to approximately 4 seconds).

In certain embodiments, the isochronic wave comprises a carrier wave [e.g., a periodic wave having a substantially constant frequency (e.g., ranging from 5 to 15 Hz; e.g., ranging from approximately 7 to approximately 13 Hz; e.g., a frequency range matching an alpha brain wave frequency range; e.g., approximately 10 Hz)] modulated by an envelope function having one or more low-amplitude sub-intervals [e.g., a periodic envelope function (e.g., a square wave; e.g., a 0.5 Hz square wave); e.g., the one or more low-amplitude sub-intervals having a duration of greater than or approximately equal to two seconds; e.g., the one or more low-amplitude sub-intervals having a duration of approximately two seconds].

In certain embodiments, the isochronic wave is also a transformed time-varying wave. In certain embodiments, the isochronic wave comprises a clipped wave. In certain embodiments, the waveform of the electronic drive signal comprises a transformed time-varying wave having a functional form corresponding to a carrier wave within an envelope {e.g., wherein the transformed-time varying wave is the carrier wave and is further modulated by an envelope [e.g., wherein the envelope is a sinusoidal wave; e.g., wherein the envelope has a monotonically increasing (in time) amplitude (e.g., wherein the envelope has a functional form corresponding to an increasing (in time) exponential)]; e.g., wherein the transformed time-varying wave is the envelope that modulates a carrier wave [e.g., wherein the carrier wave is a periodic wave (e.g., a sinusoidal wave; e.g., a square wave; e.g., a sawtooth wave)(e.g., having a higher frequency than the envelope)]}. In certain embodiments, a functional form of the waveform of the electronic drive signal is based on one or more recorded natural sounds (e.g., running water; e.g., ocean waves; e.g., purring; e.g., breathing; e.g., chanting; e.g., gongs; e.g., bells).

In certain embodiments, the method comprises receiving an electronic response signal from a monitoring device (e.g., directly from and/or to the monitoring device; e.g., via one or more intermediate server(s) and/or computing device(s))(e.g., a wearable monitoring device; e.g., a personal computing device; e.g., a fitness tracker; e.g., a heart-rate monitor; e.g., an electrocardiograph (EKG) monitor; e.g., an electroencephalography (EEG) monitor; e.g., an accelerometer; e.g., a blood-pressure monitor; e.g., a galvanic skin response (GSR) monitor) and), and wherein the controlling the waveform of the electronic drive signal comprises adjusting and/or selecting the waveform in response to (e.g., based on) the received electronic response signal.

In certain embodiments, the method comprises recording usage data (e.g., parameters such as a record of when the device was used, duration of use, etc.) and/or one or more biofeedback signals for a human subject [e.g., using one or more sensors, each operable to measure and record one or more biofeedback signals (e.g., a galvanic skin response (GSR) sensor; e.g., a heart-rate monitor; e.g., an accelerometer)] [e.g., storing and/or providing the recorded usage data and/or biofeedback signals for further processing and/or transmission to an external computing device, e.g., for computation (e.g., using a machine learning algorithm that receives the one or more biofeedback signals as input, along with, optionally, user reported information) and display of one or more performance metrics (e.g., a cognition, performance or learning index) to a subject].

In certain embodiments, the method comprises automatically modulating and/or selecting the waveform of the electronic drive signal in response to (e.g., based on) the recorded usage data and/or biofeedback signals (e.g., using a machine learning algorithm that receives the one or more biofeedback signals as input, along with, optionally, user reported information, to optimize the waveform output).

In certain embodiments, a level [e.g., amplitude (e.g., a force; e.g., a displacement)] of at least a portion of the mechanical wave is (e.g., modulated and/or selected) based on activation thresholds of one or more target cells and/or proteins (e.g., mechanoreceptors (e.g., C tactile afferents); e.g., nerves; e.g., sensory thresholds corresponding to a level of tactile sensation) [e.g., wherein the one or more controller boards modulate the waveform output based on sub-activation thresholds (e.g., accounting for the response of the mechanical transducers)].

In certain embodiments, an amplitude of the mechanical wave corresponds to a displacement ranging from 1 micron to 10 millimeters (e.g., approximately 25 microns)(e.g., wherein the amplitude is adjustable over the displacement ranging from 1 micron to 10 millimeters)[e.g., wherein the amplitude corresponds to a force of approximately 0.4N] [e.g., thereby matching the amplitude to activation thresholds of C tactile afferents].

In another aspect, the disclosure is directed to a method of improving focus, concentration, learning capacity, visual memory, new learning, sustained attention, cognition and/or interoception in a human by providing transcutaneous mechanical stimulation (e.g., non-invasive mechanical stimulation) to the subject via a stimulation device (e.g., a wearable device), the method comprising: generating a mechanical wave by a mechanical transducer of the stimulation device in response to an applied electronic drive signal; controlling a waveform of the electronic drive signal by a controller board (e.g., a controller board of the stimulation device; e.g., a remote controller board); and delivering the mechanical wave to a body location of the subject via the stimulation device, wherein the body location is in proximity to a temporal bone of the subject (e.g., wherein the temporal bone lies directly beneath the body location), thereby providing the transcutaneous mechanical stimulation to the subject.

In another aspect, the disclosure is directed to a method of improving focus, concentration, learning capacity, visual memory, new learning, sustained attention, cognition and/or interoception in a human by providing transcutaneous mechanical stimulation (e.g., non-invasive mechanical stimulation) to one or more nerves of the subject via a stimulation device (e.g., a wearable device), the method comprising: generating a mechanical wave by a mechanical transducer of the stimulation device in response to an applied electronic drive signal; controlling a waveform of the electronic drive signal by a controller board (e.g., of the stimulation device; e.g., a remote controller board); and delivering the mechanical wave to a body location of the subject via the wearable stimulation device, thereby stimulating the one or more nerves, wherein the one or more nerves comprise(s) a cranial nerve (e.g., vagus nerve; e.g., trigeminal nerve; e.g., facial nerve) of the subject.

In another aspect, the disclosure is directed to a method improving focus, concentration, learning capacity, visual memory, new learning, sustained attention, cognition and/or interoception in a human by providing transcutaneous mechanical stimulation (e.g., non-invasive mechanical stimulation) to one or more nerves and/or mechanoreceptors of the subject via a stimulation device (e.g., a wearable device), the method comprising: generating a mechanical wave by a mechanical transducer of the stimulation device in response to an applied electronic drive signal; controlling a waveform of the electronic drive signal by a controller board (e.g., a controller board of the wearable stimulation device; e.g., a remote controller board), wherein the waveform comprises a frequency component ranging from approximately 5 Hz to 15 Hz (e.g., approximately 10 Hz; e.g., ranging from approximately 7 Hz to approximately 13 Hz; e.g., a frequency range matching an alpha brain wave frequency); and delivering the mechanical wave to a body location of the subject via the stimulation device, thereby providing the transcutaneous mechanical stimulation of the one or more nerves and/or mechanoreceptors of the subject.

In another aspect, the disclosure is directed to a method of improving focus, concentration, learning capacity, visual memory, new learning, sustained attention, cognition and/or interoception in a human by providing transcutaneous mechanical stimulation (e.g., non-invasive mechanical stimulation) to the subject via a stimulation device (e.g., a wearable device), the method comprising: generating a mechanical wave by a mechanical transducer of the stimulation device in response to an applied electronic drive signal; receiving an electronic response signal from a monitoring device (e.g., a wearable monitoring device) operable to monitor one or more physiological signals from the subject and generate, in response to the one or more physiological signals from the subject, the electronic response signal (e.g., wherein the electronic response signal is received directly from the monitoring device; e.g., wherein the electronic response signal is received from the wearable monitoring device via one or more intermediate servers and/or processors); responsive to the receiving the electronic response signal, controlling, via a controller board (e.g., a controller board of the stimulation device; e.g., a remote controller board), a waveform of the electronic drive signal to adjust and/or select the waveform based at least in part on the received electronic response signal; and delivering the mechanical wave to a body location of the subject via the stimulation device, thereby providing the transcutaneous mechanical stimulation to the subject.

In another aspect, the disclosure is directed to a method of improving focus, concentration, learning capacity, visual memory, new learning, sustained attention, cognition and/or interoception in a human by providing transcutaneous mechanical stimulation (e.g., non-invasive mechanical stimulation) to the subject via a stimulation device (e.g., a wearable device), the method comprising: (a) generating a mechanical wave by a mechanical transducer of the stimulation device in response to an applied electronic drive signal; (b) accessing and/or receiving [e.g., by a processor of a computing device, of and/or in communication with the stimulation device, e.g., an intermediate server and/or processor (e.g., of a mobile computing device in communication with the stimulation device)] subject response data (e.g., entered by the subjects themselves or biofeedback data recorded via sensors) and/or initialization setting data [e.g., physical characteristics of the subject (e.g., age, height, weight, gender, body-mass index (BMI), and the like); e.g., activity levels (e.g., physical activity levels); e.g., biofeedback data recorded by one or more sensors (e.g., included within the device and/or external to and in communication with the device)(e.g., a heart rate; e.g., a galvanic skin response; e.g., physical movement (e.g., recorded by an accelerometer)); e.g., results of a preliminary survey (e.g., entered by the subject themselves, e.g., via a mobile computing device, an app, and/or online portal; e.g., provided by a therapist/physician treating the human)]; (c) responsive to the accessed and/or received subject response data and/or initialization setting data, controlling, via a controller board (e.g., a controller board of the stimulation device; e.g., a remote controller board), a waveform of the electronic drive signal to adjust and/or select the waveform based at least in part on the subject response data and/or initialization setting data (e.g., using a machine learning algorithm that receives one or more biofeedback signals as input, along with, optionally, user reported information, to optimize the waveform output); and (d) delivering the mechanical wave to a body location of the subject via the stimulation device, thereby providing the transcutaneous mechanical stimulation to the subject.

In certain embodiments, step (b) comprises receiving and/or accessing subject response data [e.g., results of a survey recorded for the subject (e.g., entered by the subject themselves, e.g., via a mobile computing device, an app, and/or online portal; e.g., provided by a therapist/physician treating the human); e.g., biofeedback data recorded by one or more sensors (e.g., included within the device and/or external to and in communication with the device)(e.g., a heart rate; e.g., a galvanic skin response; e.g., physical movement (e.g., recorded by an accelerometer))] provided following their receipt of a round (e.g., a duration) of the transcutaneous mechanical stimulation provided by the stimulation device; and step (c) comprises controlling the waveform of the electronic drive signal based at least in part on the subject feedback, thereby modifying the transcutaneous mechanical stimulation provided to the subject based on subject response data.

In another aspect, the disclosure is directed to a method of improving focus, concentration, learning capacity, visual memory, new learning, sustained attention, cognition and/or interoception in a human by providing transcutaneous mechanical stimulation (e.g., non-invasive mechanical stimulation) to the subject via a stimulation device (e.g., a wearable device), the method comprising: generating a first mechanical wave by a first mechanical transducer of the stimulation device in response to a first applied electronic drive signal; controlling a first waveform of the first electronic drive signal by a controller board (e.g., a controller board of the stimulation device; e.g., a remote controller board); delivering the first mechanical wave to a first body location (e.g., on a right side; e.g., a location behind a right ear) of the subject via the stimulation device; generating a second mechanical wave by a second mechanical transducer of the stimulation device in response to a second applied electronic drive signal; controlling a second waveform of the second electronic drive signal by the controller board; and delivering the second mechanical wave to a second body location (e.g., on a left side; e.g., a location behind a left ear) of the subject via the stimulation device, thereby providing the transcutaneous mechanical stimulation to the subject.

In another aspect, the disclosure is directed to a method of improving focus, concentration, learning capacity, visual memory, new learning, sustained attention, cognition and/or interoception in a human by providing transcutaneous mechanical stimulation (e.g., non-invasive mechanical stimulation) to the subject via a stimulation device (e.g., a wearable device), the method comprising: generating a first mechanical wave by a first mechanical transducer of the stimulation device in response to an applied electronic drive signal; controlling a waveform of the first electronic drive signal by a controller board (e.g., a controller board of the stimulation device; e.g., a remote controller board); delivering the first mechanical wave to a first body location (e.g., on a right side; e.g., a location behind a right ear) of the subject via the stimulation device; generating a second mechanical wave by a second mechanical transducer of the stimulation device in response to the applied electronic drive signal; delivering the second mechanical wave to a second body location (e.g., on a left side; e.g., a location behind a left ear) of the subject via the stimulation device, thereby providing the transcutaneous mechanical stimulation to the subject.

In another aspect, the disclosure is directed to a method of improving focus, concentration, learning capacity, visual memory, new learning, sustained attention, cognition and/or interoception in a human by providing transcutaneous mechanical stimulation (e.g., non-invasive mechanical stimulation) to one or more nerves and/or mechanoreceptors of the subject via a stimulation device (e.g., a wearable device), in combination with one or more rounds of a therapy [e.g., psychotherapy; e.g., exposure therapy; e.g., cognitive behavioral therapy (CBT); e.g., acceptance and commitment therapy (ACT)] the method comprising: generating a mechanical wave by a mechanical transducer of the stimulation device in response to an applied electronic drive signal; controlling a waveform of the electronic drive signal by a controller board (e.g., a controller board of the wearable stimulation device; e.g., a remote controller board); and delivering the mechanical wave to a body location of the subject via the stimulation device at one or more times each in proximity to and/or during a round of the therapy received by the subject thereby providing the transcutaneous mechanical stimulation of the one or more nerves and/or mechanoreceptors of the subject in combination with one or more rounds of the therapy.

In another aspect, the disclosure is directed to a method of improving focus, concentration, learning capacity, visual memory, new learning, sustained attention, cognition and/or interoception in a human by stimulating one or more nerves and/or mechanoreceptors of the subject (e.g., a human subject), the method comprising: using the device method comprising: using the device articulated in any of paragraphs [007]-[082], for stimulation of the one or more nerves and/or mechanoreceptors of the subject.

In another aspect, the disclosure is directed to a method of improving focus, concentration, learning capacity, visual memory, new learning, sustained attention, cognition and/or interoception in a human by stimulating one or more nerves of the human subject using a transcutaneous, neuromodulation device [e.g., a wearable device; e.g., a non-invasive device (e.g., not comprising any components that penetrate skin)], the device comprising one or more transducers (e.g., mechanical transducers), a battery, connectors, and one or more controller boards, wherein the one or more controller boards control waveform output through the connectors and the transducers, and wherein the transducers transcutaneously applied stimulates the one or more nerves, the method comprising: contacting the one or more transducers of the device to the human subject, generating the waveform output signal, activating the transducers using the waveform output signal (e.g., by applying the waveform output signal to the transducers to generate a mechanical wave), and stimulating the one or more nerves of the human subject, wherein the waveform output comprises an isochronic wave.

In another aspect, the disclosure is directed to a method of improving focus, concentration, learning capacity, visual memory, new learning, sustained attention, cognition and/or interoception in a human by stimulating one or more mechanoreceptors of the human subject using transcutaneous stimulation device [e.g., a wearable device; e.g., a non-invasive device (e.g., not comprising any components that penetrate skin)], the device comprising one or more mechanical transducers, a battery, connectors, and one or more controller boards, wherein the one or more controller boards control waveform output through the connectors and the one or more mechanical transducers, and wherein the one or more mechanical transducers transcutaneously applied stimulate the one or more mechanoreceptors, the method comprising: contacting the one or more mechanical transducers of the device to the human subject, generating the waveform output signal, activating the mechanical transducers using the waveform output signal (e.g., by applying the waveform output signal to the transducers to generate a mechanical wave), and stimulating the one or more mechanoreceptors of the human subject, wherein the waveform output comprises an isochronic wave.

In another aspect, the disclosure is directed to a method of adjusting (e.g., controlling) a level of a stress hormone [e.g., cortisol (e.g., reducing a cortisol level); e.g., oxytocin (e.g., increasing an oxytocin level); e.g., serotonin (e.g., increasing a serotonin level)] in a subject, the method comprising transcutaneously delivering mechanical stimulation to the subject using a mechanical wave having a vibrational waveform selected to reduce the level of the stress hormone in the subject upon and/or following the delivering of the mechanical wave to the subject.

In another aspect, the disclosure is directed to a kit comprising the device of any one of the aspects and embodiments described herein and a label indicating that the device is to be used for improving focus, concentration, learning capacity, visual memory, new learning, sustained attention, cognition and/or interoception in a human.

In another aspect, the disclosure is directed to a transcutaneous neuromodulation device [e.g., a wearable device; e.g., a non-invasive device (e.g., not comprising any components that penetrate skin)] for improving focus, concentration, learning capacity, visual memory, new learning, sustained attention, cognition and/or interoception in a human by promoting nerve stimulation through mechanical vibration, comprising: one or more mechanical transducers, a battery, and a controller board, wherein the transducer, battery and controller board are in communication (e.g., through one or more connectors; e.g., wirelessly), and wherein the controller board controls waveform output through the transducer, thereby producing a mechanical vibration.

Elements of embodiments involving one aspect of the disclosure (e.g., compositions, e.g., systems, e.g., methods) can be applied in embodiments involving other aspects of the disclosure, and vice versa.

### BRIEF DESCRIPTION OF THE DRAWINGS

The foregoing and other objects, aspects, features, and advantages of the present disclosure will become more apparent and better understood by referring to the following description taken in conjunction with the accompanying drawings, in which:

FIG. 1 shows an image of the COVE® device used in the studies shown and described herein.

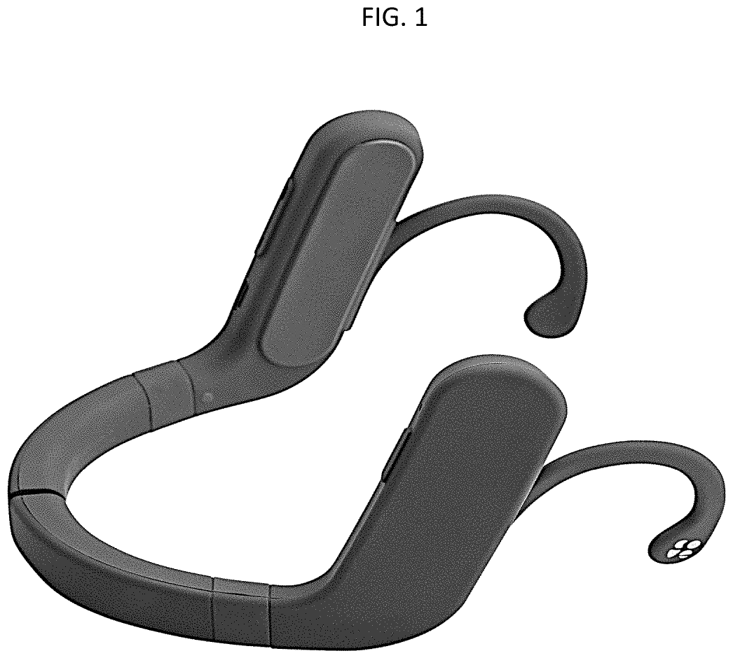

FIG. 2 shows another image of the COVE® device used in the studies shown and described herein.

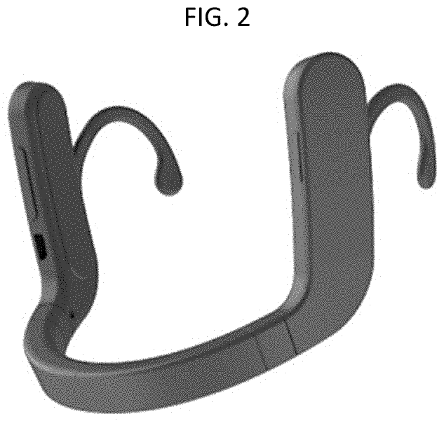

FIG. 3 shows examples of waveforms used in accordance with at least some embodiments of the present invention described herein.

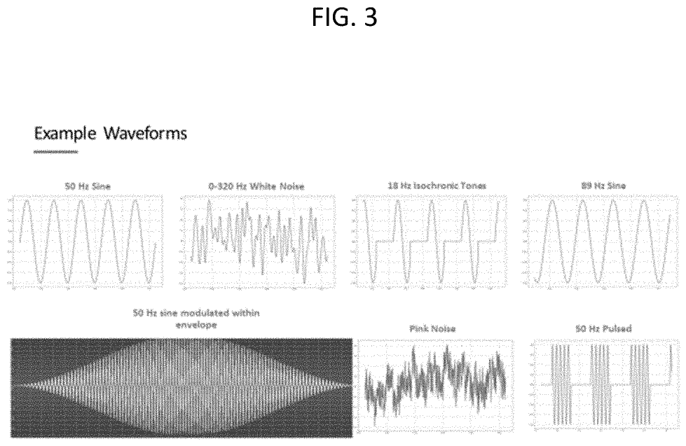

FIG. 4 is a table showing demographic information regarding the participants of one of the studies shown and described herein.

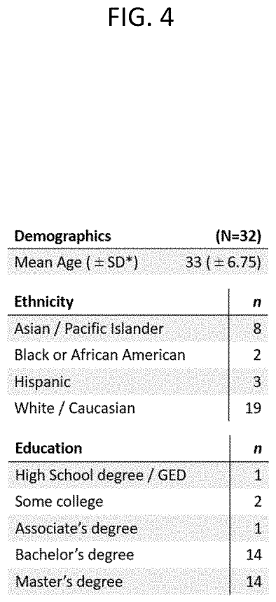

FIG. 5 is a timeline of key aspects of the one of the study shown and described herein.

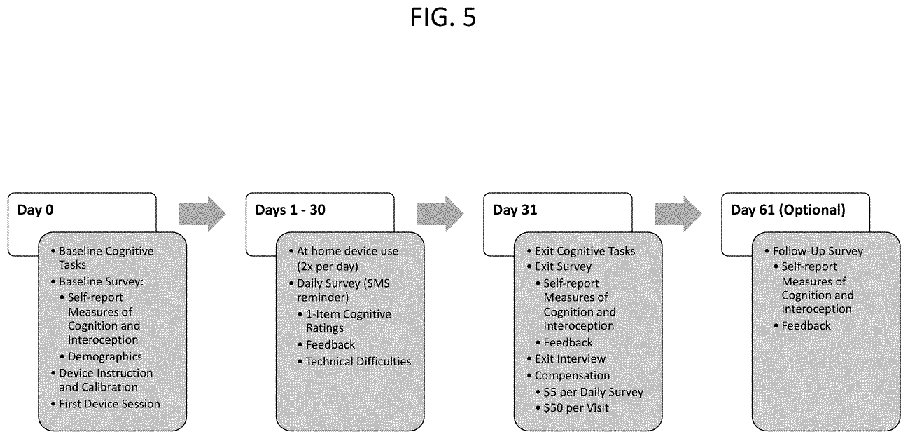

FIG. 6 is a table summarizing the PAL Task outcome measures and related details.

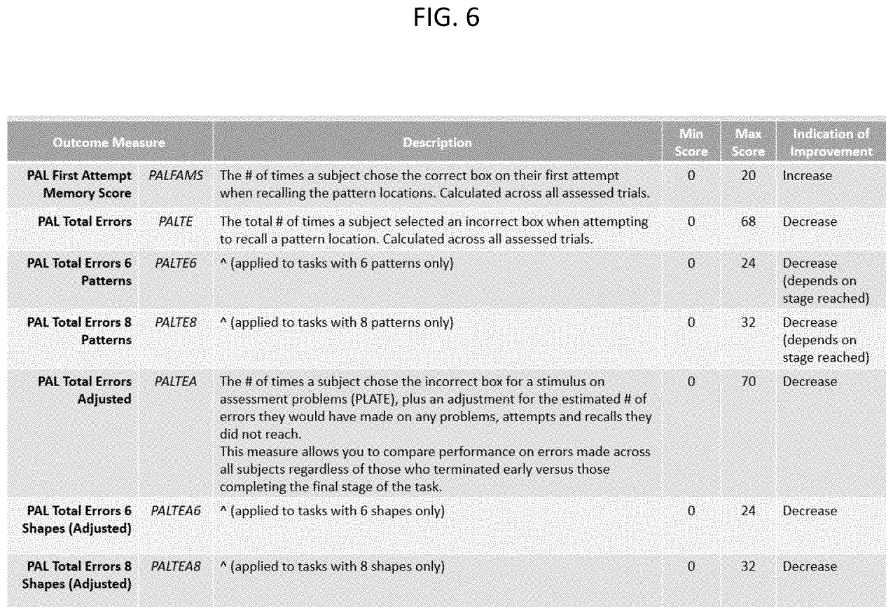

FIG. 7 is a table summarizing the RVP Task outcome measures and related details.

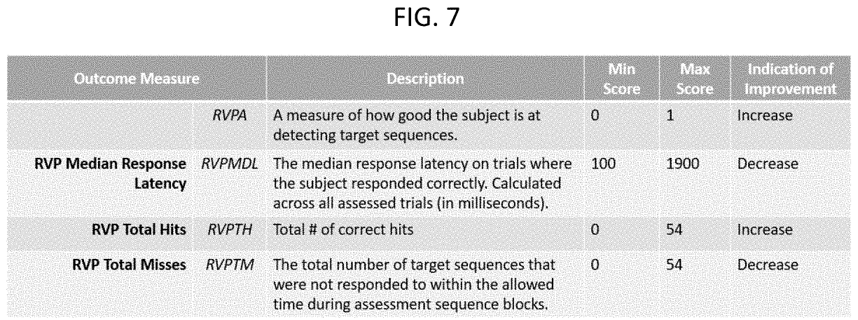

FIG. 8 is an image defining the dimensions of interoception assessed (MAIA).

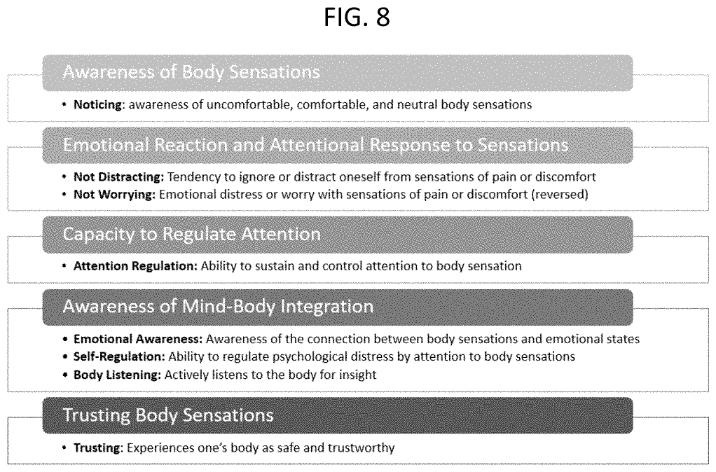

FIG. 9 is a chart detailing the 1-Item Cognitive Rating questions related to focus, concentration, and learning capacity, asked of participants of the study disclosed herein.

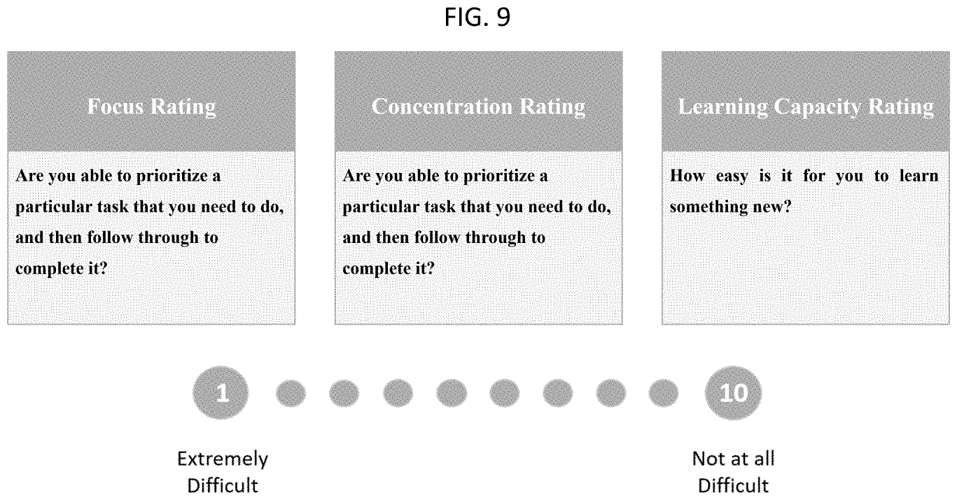

FIG. 10 is a graph of the Average PAL Task First Attempt Memory Scores (PALFAMS) for a 10 Hz sample.

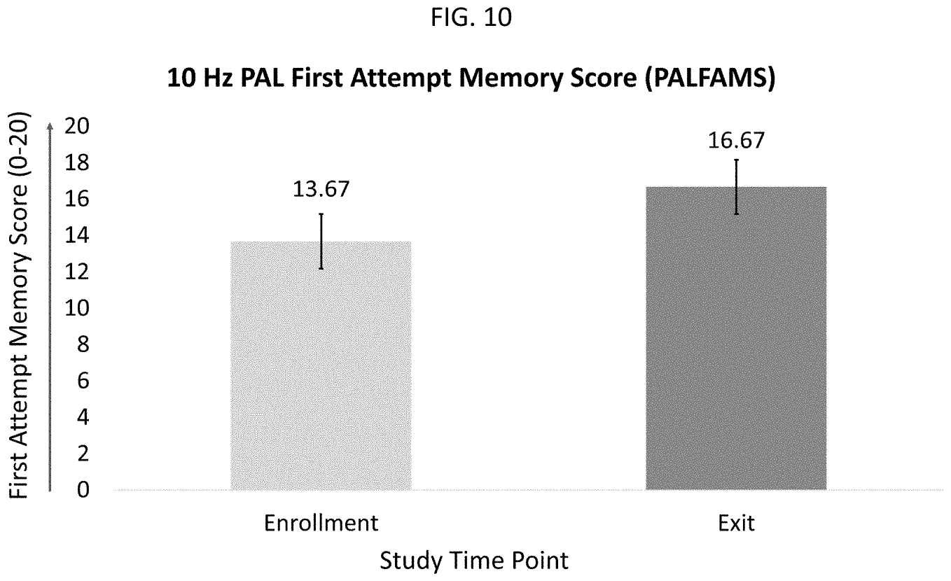

FIG. 11 is a graph of the Average PAL Task Total Errors vs Total Errors Adjusted for a 10 Hz sample.

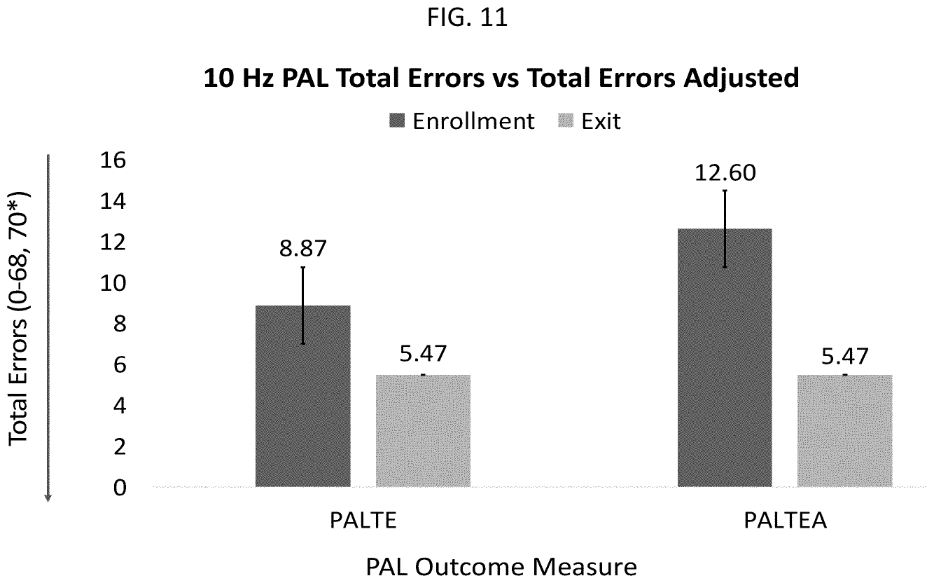

FIG. 12 is a graph of the Average PAL Task Total Errors vs Total Errors Adjusted (6 Patterns) for a 10 Hz sample.

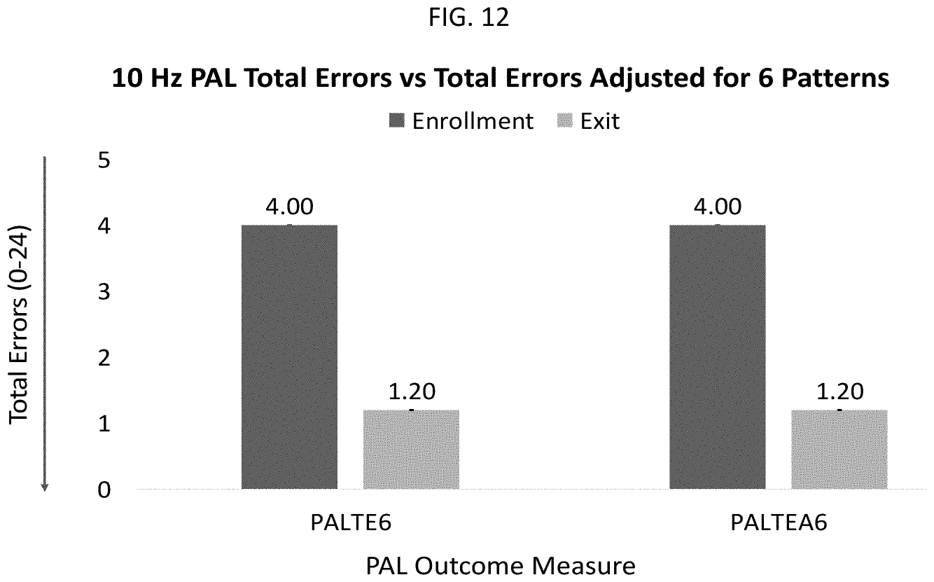

FIG. 13 is a graph of the Average PAL Task Total Errors vs Total Errors Adjusted (8 Patterns) for a 10 Hz sample.

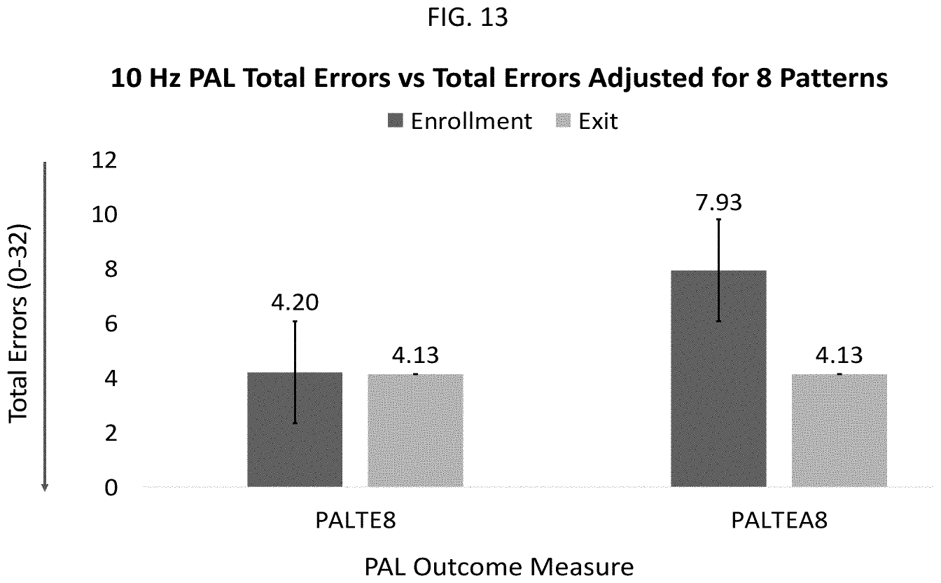

FIG. 14 is a graph of the Average Proficiency for Detecting Target Sequences in the RVP Task (RVPA) for a 10 Hz sample.

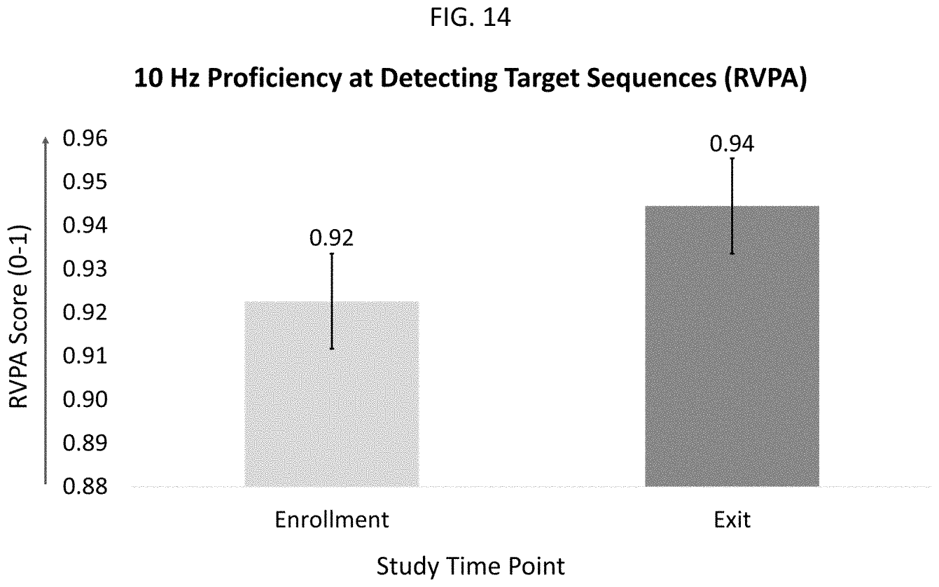

FIG. 15 is a graph of the Average RVP Task Median Response Latency (RVPMDL) for a 10 Hz sample.

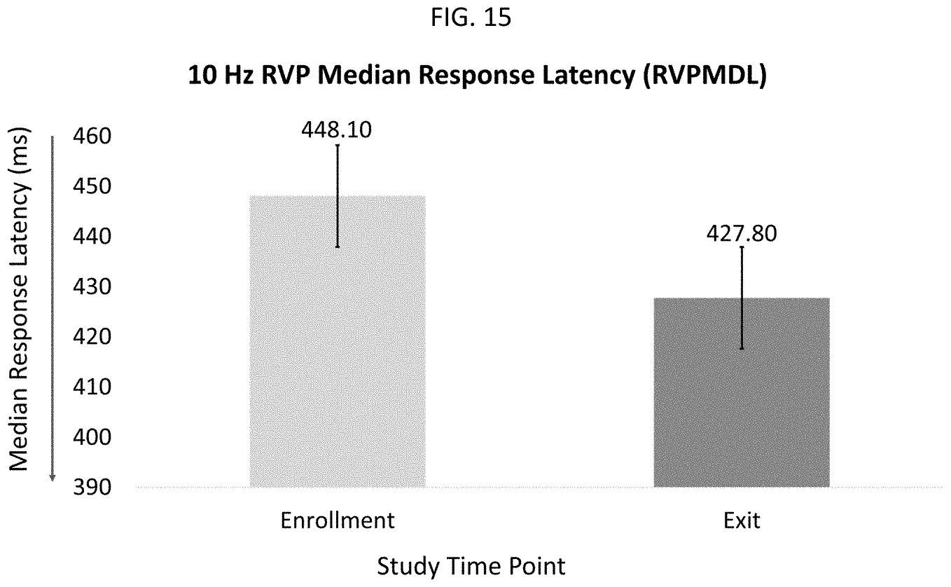

FIG. 16 is a graph of the Average RVP Task Total Hits (RVPTH) for a 10 Hz sample.

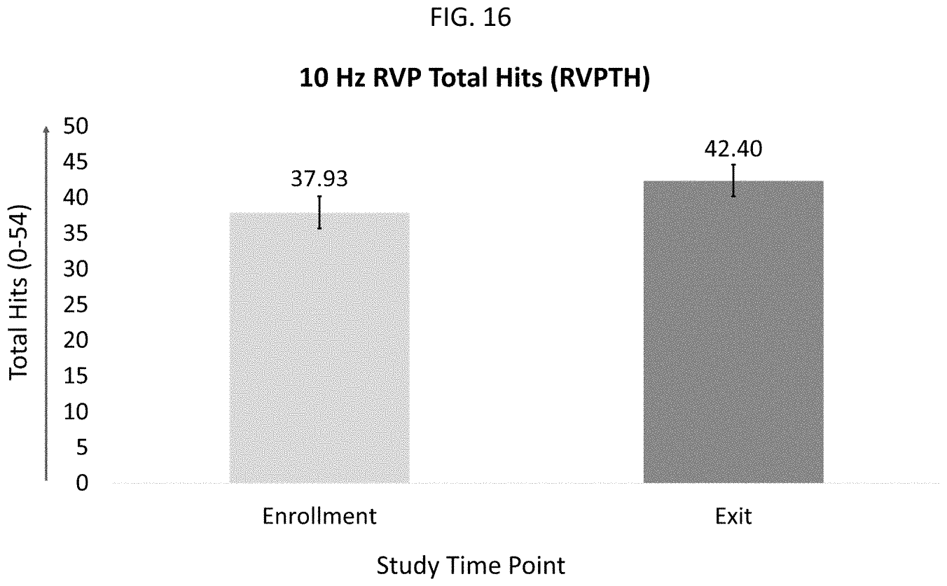

FIG. 17 is a graph of the Average RVP Task Total Misses (RVPTM) for a 10 Hz sample.

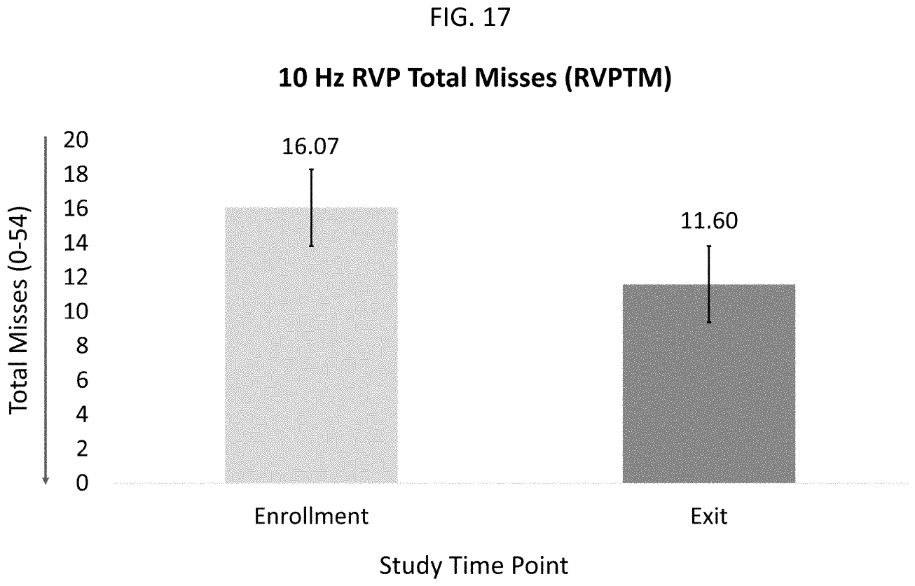

FIG. 18 is a graph of Average Focus Ratings for a 10 Hz sample.

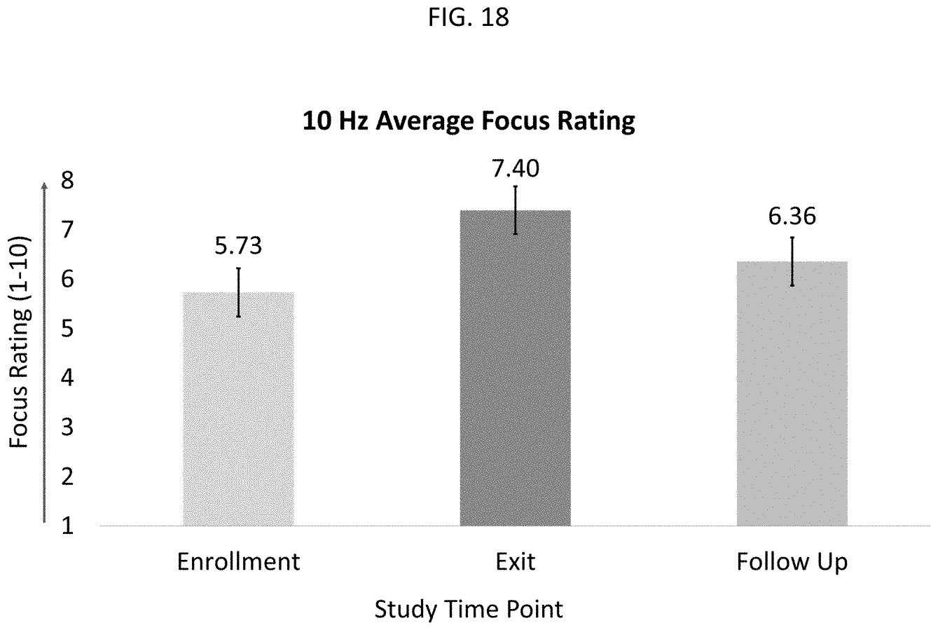

FIG. 19 is a graph of Average Concentration Ratings for a 10 Hz sample.

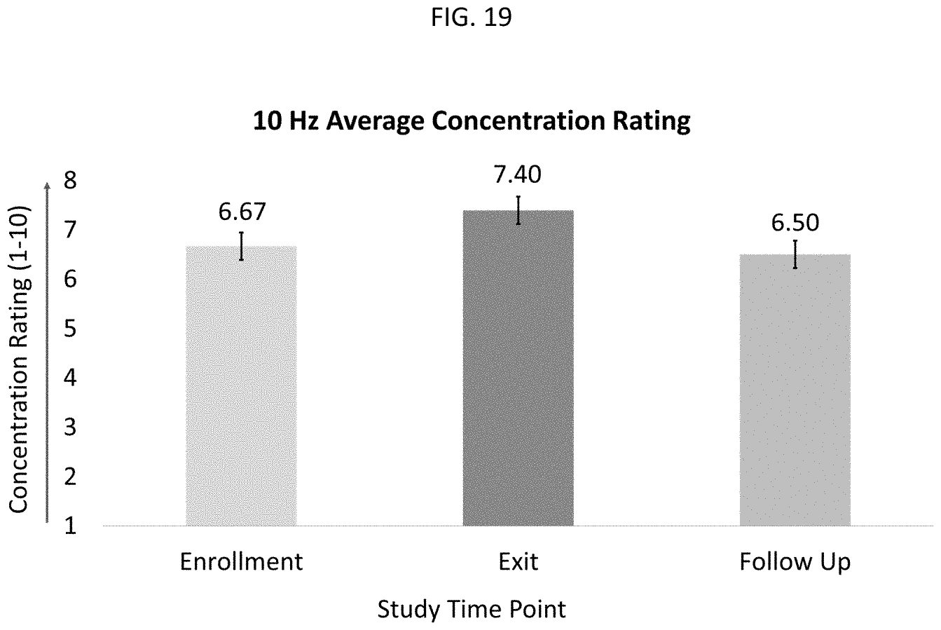

FIG. 20 is a graph of Average Learning Capacity Ratings for a 10 Hz sample.

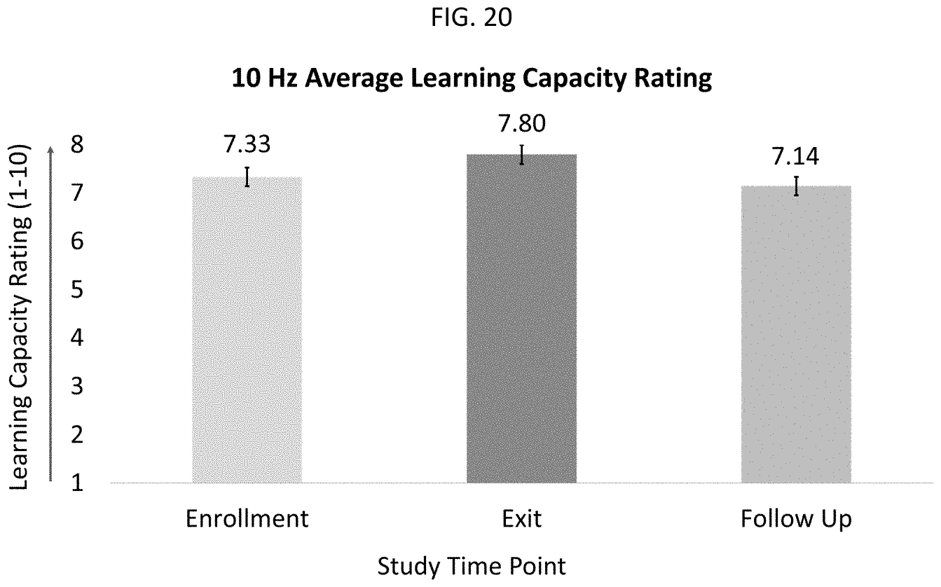

FIG. 21 is a graph of the Average Cognitive Rating Scores for a 10 Hz sample.

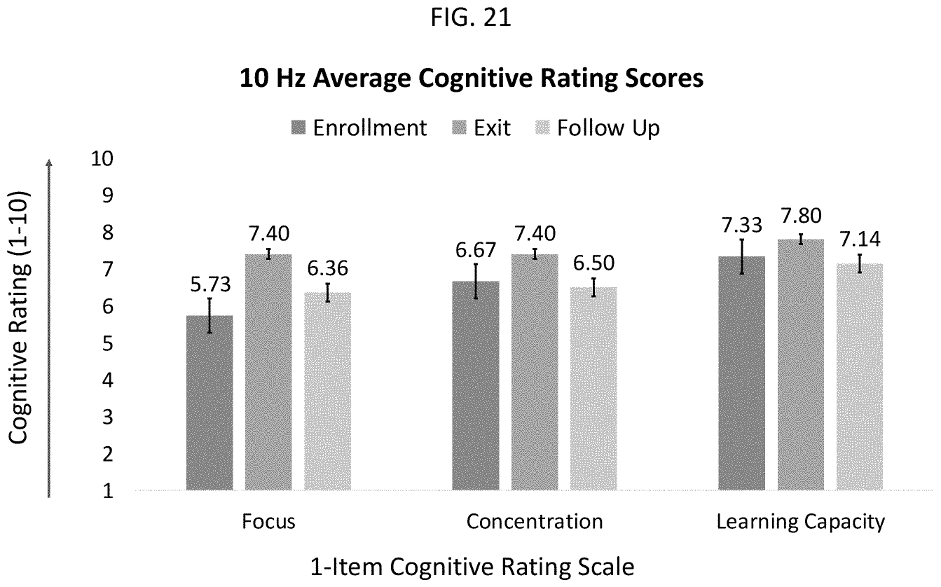

FIG. 22 is a graph of the Average MAIA Scores for a 10 Hz sample.

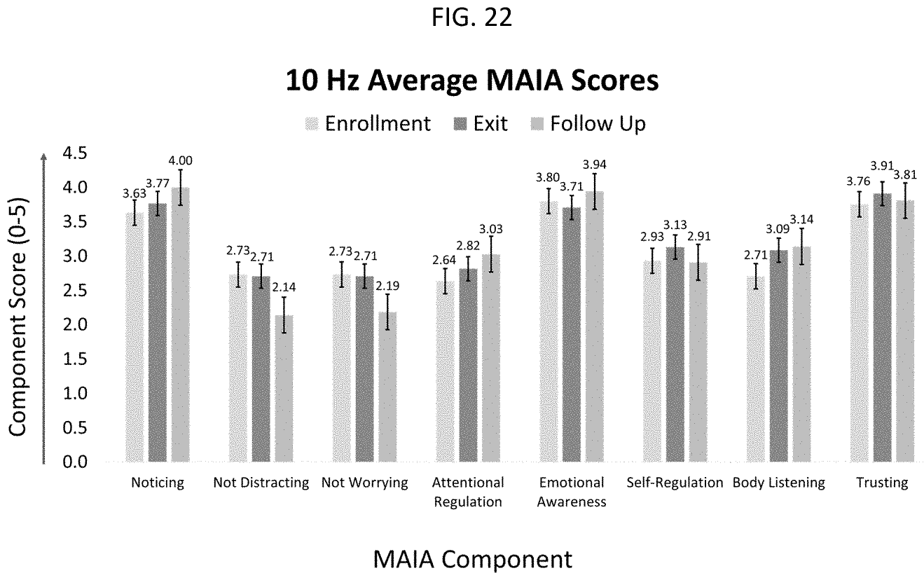

FIG. 23 is a graph of the Average Percent Change in MAIA Scores for a 10 Hz sample.

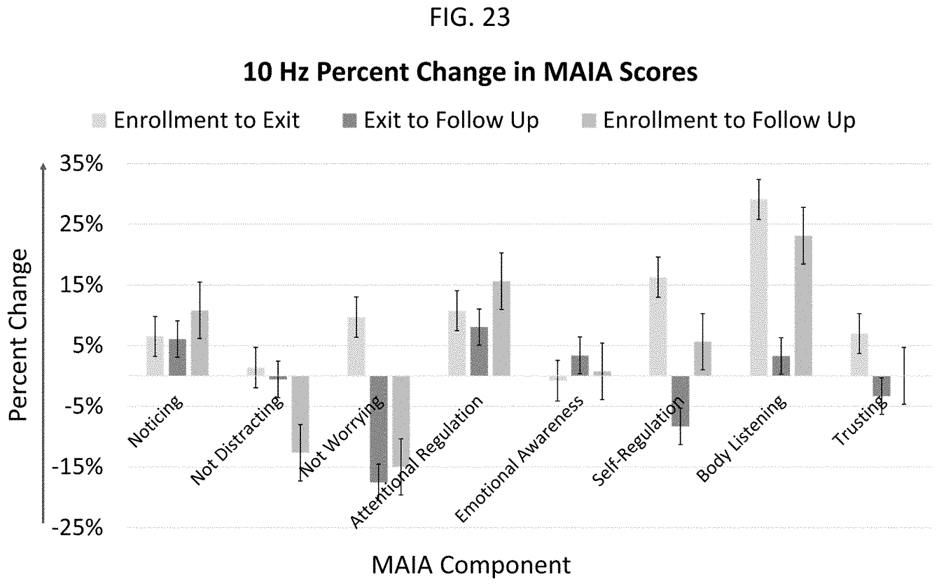

FIG. 24 is a graph of the Average PAL Task First Attempt Memory Scores (PALFAMS) for a 20 Hz sample.

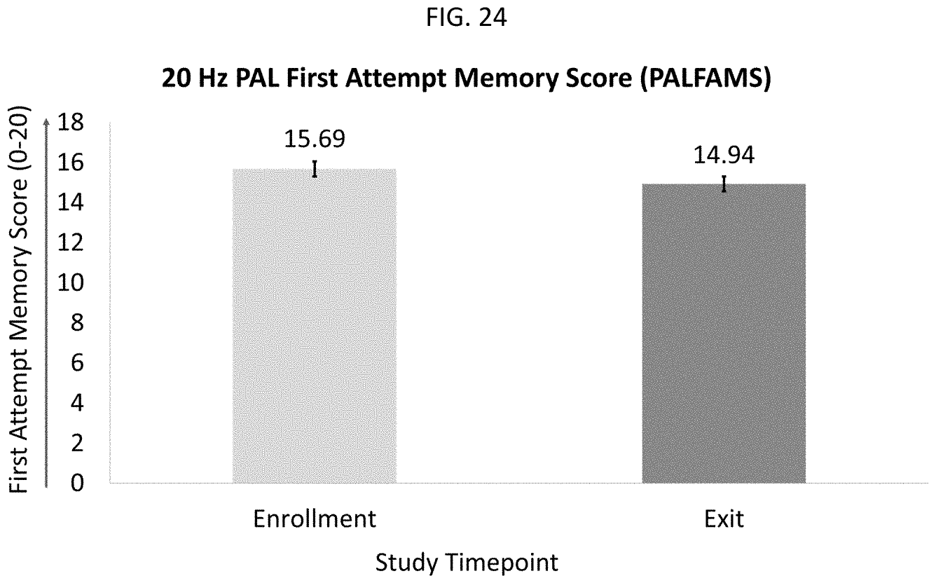

FIG. 25 is a graph of the Average PAL Total Errors vs Total Errors Adjusted for a 20 Hz sample.

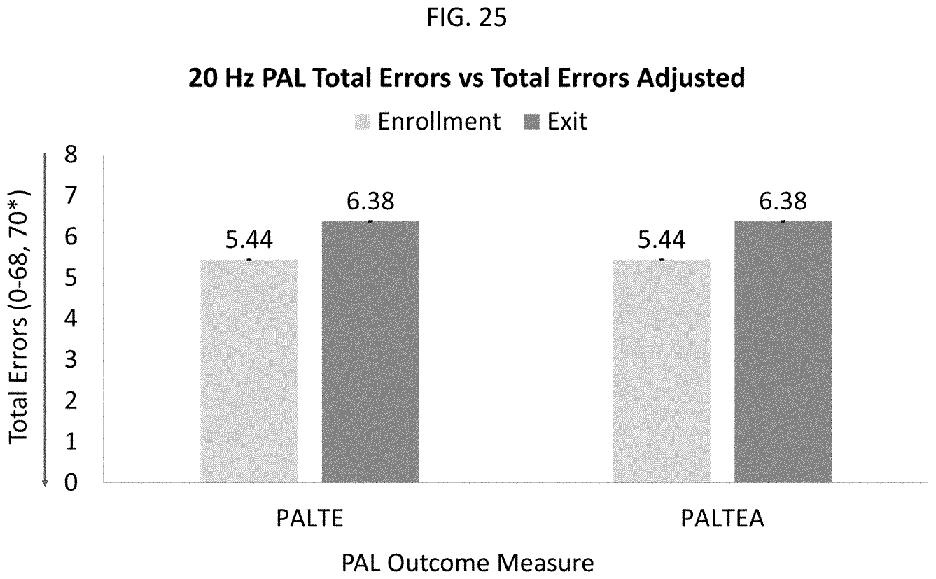

FIG. 26 is a graph of the Average Proficiency for Detecting Target Sequences in the RVP Task (RVPA) for a 20 Hz sample.

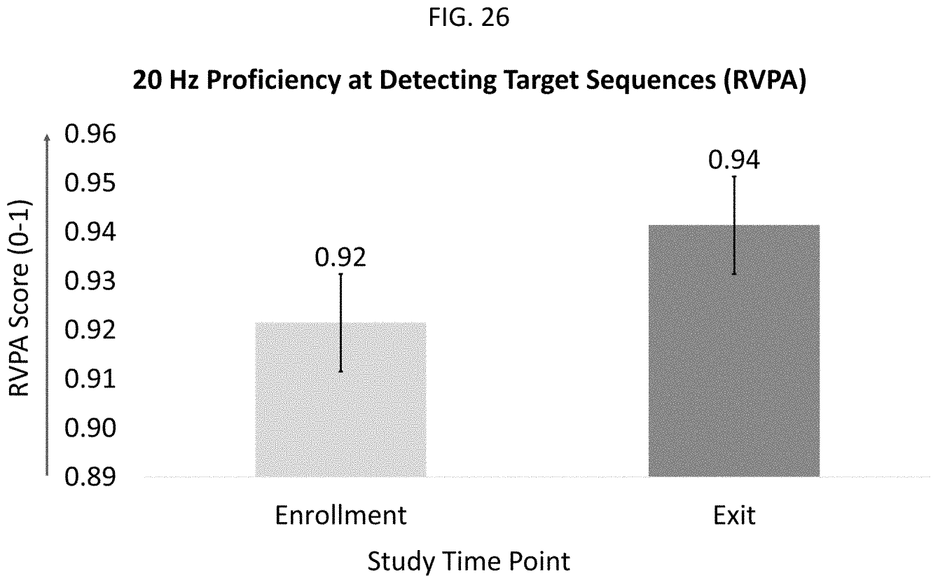

FIG. 27 is a graph of the Average RVP Task Median Response Latency (RVPMDL) for a 20 Hz sample.

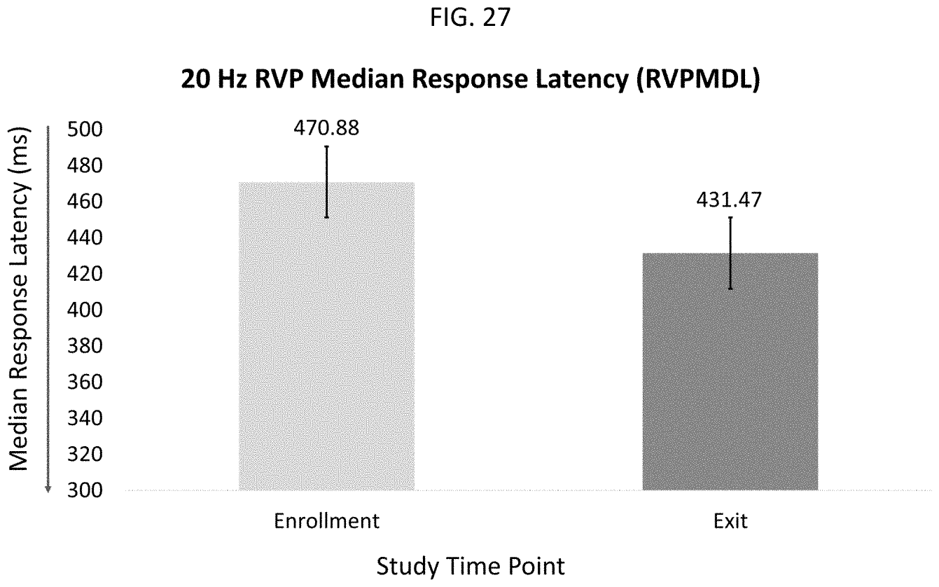

FIG. 28 is a graph of the Average RVP Task Total Hits (RVPTH) for a 20 Hz sample.

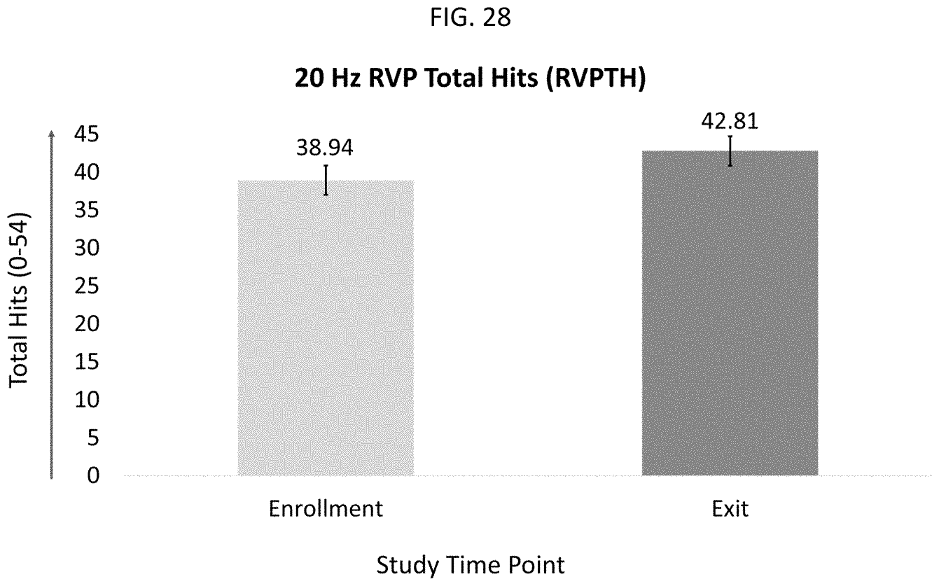

FIG. 29 is a graph of the Average RVP Task Total Misses (RVPTM) for a 20 Hz sample.

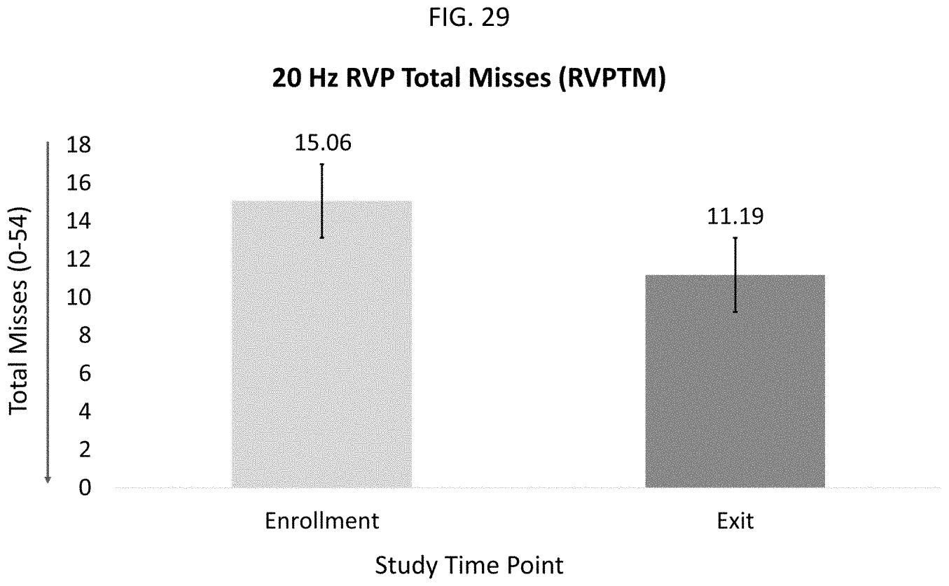

FIG. 30 is a graph of Average Focus Ratings for a 20 Hz sample.

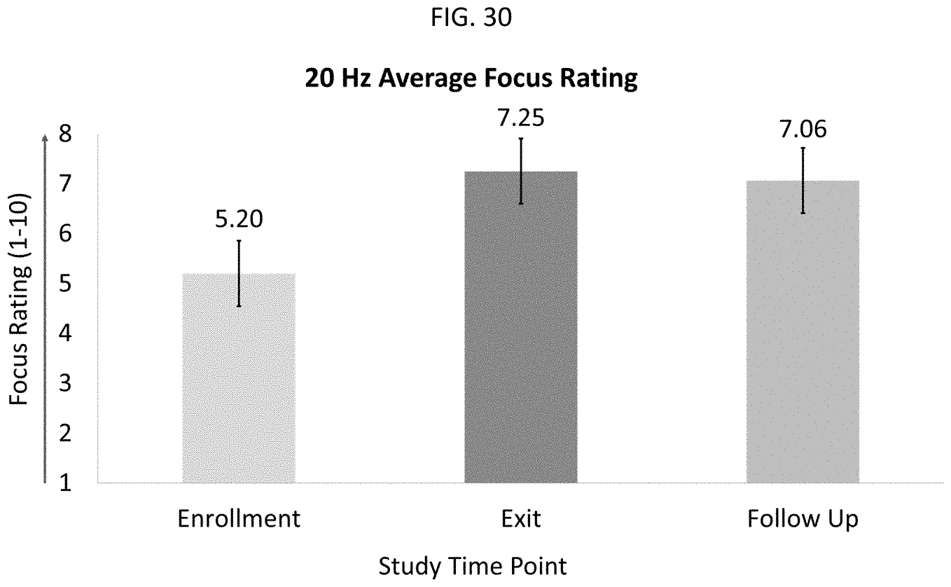

FIG. 31 is a graph of Average Concentration Ratings for a 20 Hz sample.

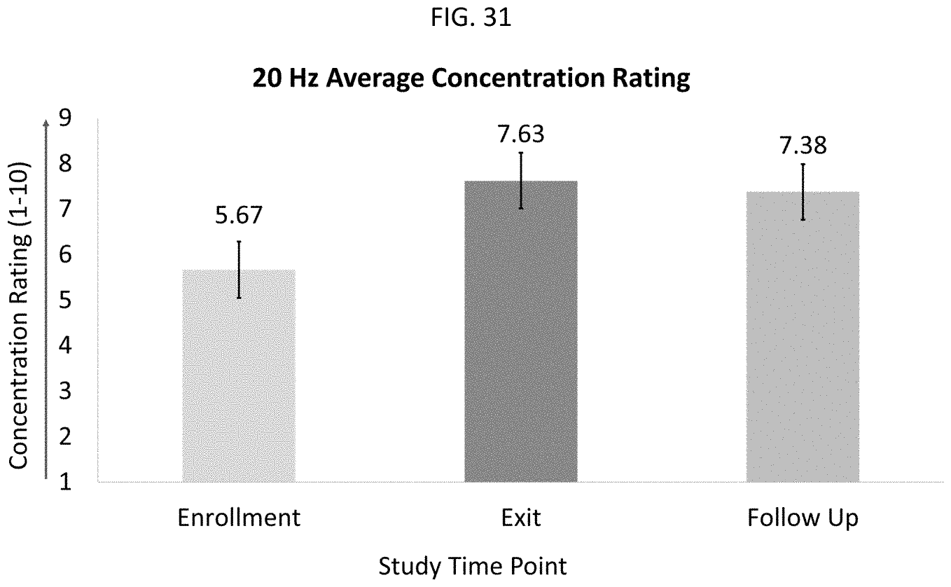

FIG. 32 is a graph of the Average Learning Capacity Ratings for a 20 Hz sample.

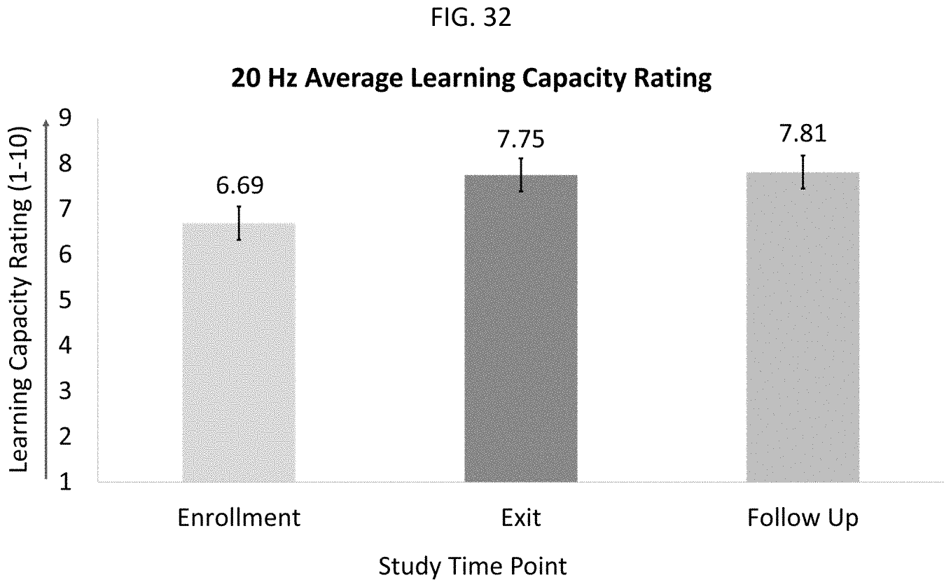

FIG. 33 is a graph of the Average Cognitive Rating Scores for a 20 Hz sample.

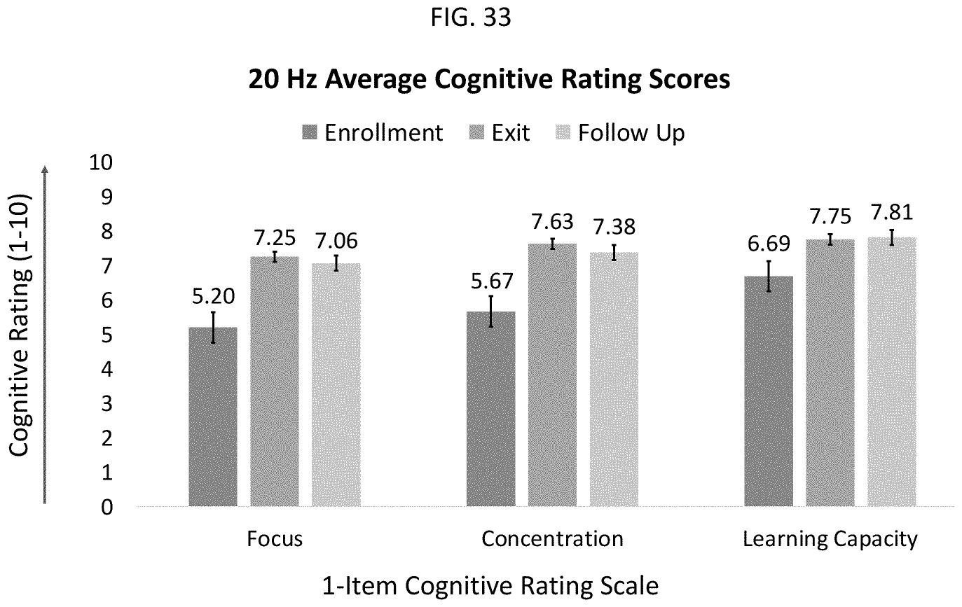

FIG. 34 is a graph of the Average MAIA Scores for a 20 Hz sample.

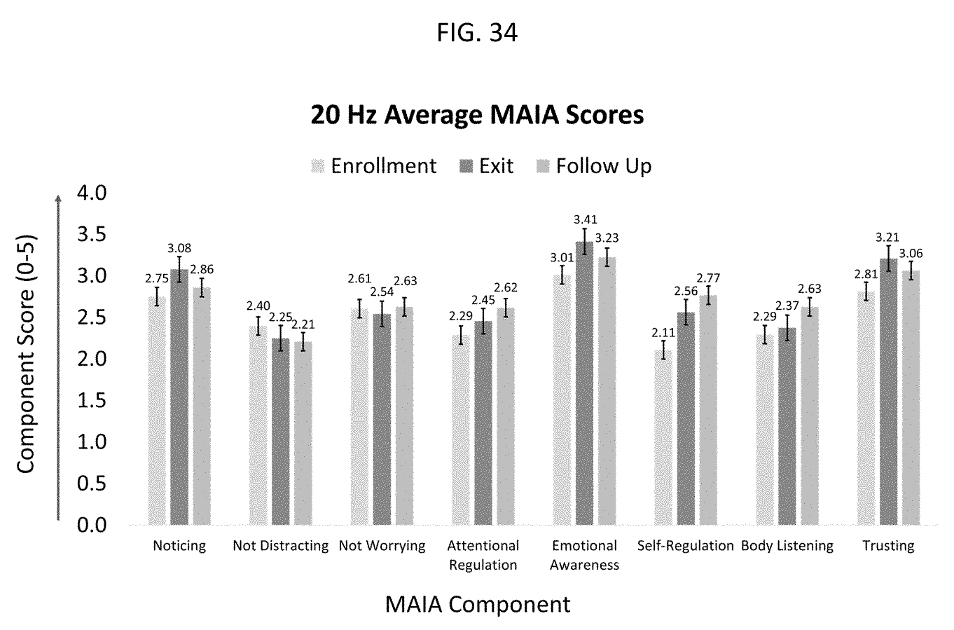

FIG. 35 is a graph of the Average Percent Change in MAIA Scores for a 20 Hz sample.

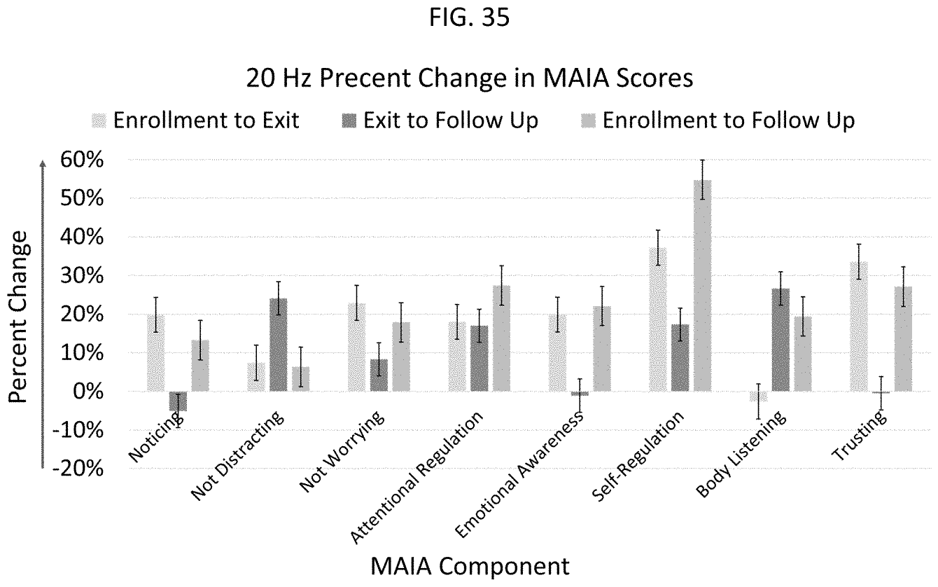

### DETAILED DESCRIPTION

A study was done to determine the effect the devices and methods disclosed herein on improving focus, concentration, learning capacity, visual memory, new learning, sustained attention, cognition and/or interoception in a human. As a result, study participants were identified and 32 participants enrolled. Study visits were completed virtually, participants were given a COVE® device, and participated in an instructional visit where the device was then calibrated prior to at home use, along with instructions to use the device two times daily. Cognitive assessments were administered pre and post 30 days of device use. The following information was obtained and tests performed.

ACS=Attention Control Scale, a 20-item self-report measure of attentional control (healthy average=˜50) used to screen individuals for study eligibility

Paired Associates Learning (PAL) Task. 8-minute cognitive task assessing visual memory and new learning.

PAL task performance results demonstrated that after 30 days of using the COVE® device, there was an observable improvement in visual memory and new learning in a 10 Hz sample (n=15). As shown in the results and FIGS provided herein, there was 39% increase in the group's visual memory and new learning, when accurately recalling pattern location (PALFAMS). Participants (n=15) also made fewer errors (PALTE), and were able to reach the highest level of the task when compared to baseline (PALTE vs PALTEA). On average, PAL task performance in a 20 Hz sample (n=16) did not improve after 30 days of using the COVE® device, indicating that visual memory and new learning were not affected by mechanical stimulation of 20 Hz.

Rapid Visual Information Processing (RVP) Task. 7-minute cognitive task assessing sustained attention.

RVP task performance results demonstrated that after 30 days of using the COVE® device, there was also an observable improvement in sustained attention for both 10 Hz and 20 Hz samples. Participants were better at detecting target sequences (RVPA), responded faster (RVPMDL), provided more accurate responses (RVPTH) and missed less target sequences (RVPTM) as indicated by the results of the aforementioned tests.

Study participants also completed self-reported 1-item cognitive ratings, in which participants are asked to rate their ability to focus, concentrate, and learn. On average, after 30 days of using the COVE® device, participants in both 10 Hz and 20 Hz samples reported an overall improvement in focus, concentration, and learning capacity. In a 10 Hz sample (n=15), Focus increased by 39%, Concentration increased by 16%, Learning Capacity increased by 7.5%. In a 20 Hz sample (n=16), Focus increased by 49%, Concentration increased by 51%, and Learning Capacity increased by 22%. On average, participants in both 10 Hz and 20 Hz samples were able to maintain their improvements in focus, for at least 30 days following the study, as focus ratings did not return to baseline at follow up.

As the results disclosed herein demonstrate, on average, after using the COVE® device for 30 days, participants in a 10 Hz sample (n=15) showed an overall improvement in cognition: visual memory, new learning, and sustained attention improved, measured by cognitive task performance; and focus, concentration and learning capacity improved, measured by self-reported 1-item cognitive ratings.

These results demonstrate that the device and methods disclosed herein provide a positive improvement in focus, concentration, learning capacity, visual memory, new learning, sustained attention, cognition and interoception.

What is claimed is:

1. A device for improving focus, concentration, learning capacity, visual memory, new learning, sustained attention, cognition and/or interoception in a human, the device comprising:
one or more mechanical transducers capable of creating transcutaneous mechanical vibrations on the body of a human;
one or more batteries;
one or more controller boards that control the output of the mechanical transducers;
wherein the one or more mechanical transducers, the one or more batteries and the one or more controller boards are in communication;
wherein the controller board controls the output of the one or more mechanical transducers, thereby producing transcutaneous mechanical vibrations for a human and wherein when the device provides transcutaneous mechanical vibrations in proximity to the temporal bone of the human's head.

2. The device of claim 1, wherein the frequency of the one or more waveform is less than 20 Hz.

3. The device of claim 1, wherein the frequency of the one or more waveforms is approximately 10 Hz.

4. The device of claim 1, wherein the one or more waveforms are isochronic.

5. The device of claim 1, wherein the device delivers mechanical vibrations in proximity to the temporal bone for at least 10 minutes per day.

6. The device of claim 5, wherein the device delivers mechanical vibrations in proximity to the temporal bone at least one time per day for a period of at least 4 weeks.

7. A device for improving focus, concentration, learning capacity, visual memory, new learning, sustained attention, cognition and/or interoception in a human, the device comprising:
one or more mechanical transducers,
one or more batteries, and
one or more sinusoidal waveforms and one or more controller boards that control at least the one or more sinusoidal waveforms output through the mechanical transducers;
wherein the one or more mechanical transducers, the one or more batteries and the one or more controller boards are in communication;
wherein the controller board controls sinusoidal waveform output through the one or more mechanical transducers, thereby producing mechanical vibrations for a human and wherein when the device is adapted to provide mechanical vibrations in proximity to the temporal bone of the human's head.

8. The device of claim 7, wherein the frequency of the one or more waveform is less than 20 Hz.

9. The device of claim 7, wherein the frequency of the one or more waveforms is approximately 10 Hz.

10. The device of claim 7, wherein the one or more waveforms are isochronic.

11. The device of claim 7, wherein the device delivers mechanical vibrations in proximity to the temporal bone for at least 20 minutes per day.

12. The device of claim 11, wherein the device delivers mechanical vibrations in proximity to the temporal bone at least 2 times per day for a period of at least 4 weeks.

13. A device for improving focus, concentration, learning capacity, visual memory, new learning, sustained attention, cognition and interoception in a human, the device comprising:
one or more mechanical transducers that create mechanical vibrations,
one or more batteries, and
one or more controller boards that control the one or more mechanical transducers;
wherein the one or more mechanical transducers, the one or more batteries and the one or more controller boards are in communication with each other;
wherein the controller board controls the output through the one or more mechanical transducers, thereby producing mechanical vibrations and wherein the device provides transcutaneous mechanical vibrations in proximity to the temporal bone of the human's head.

14. The device of claim 13, wherein the frequency is less than 20 Hz.

15. The device of claim 13, wherein the frequency is approximately 10 Hz.

16. The device of claim 13, wherein the waveforms of the mechanical vibrations are isochronic.

17. The device of claim 13, wherein the device delivers mechanical vibrations in proximity to the temporal bone for at least 20 minutes per day at least 2 times per day for a period of at least 4 weeks.

18. A method of improving focus, concentration, learning capacity, visual memory, new learning, sustained attention, cognition and/or interoception in a human, the method comprising:
delivering to a human body transcutaneous mechanical vibrations having a frequency of less than 20 Hz for at least 10 minutes per day at least 2 times per day for a period of at least 4 weeks.

19. The method of claim 18, wherein the frequency is approximately 10 Hz.

20. The method of claim 18, wherein the waveforms of the mechanical vibrations are isochronic.
